(19) United States
(12) Patent Application Publication
(10) Pub. No.: US 2019/0000326 A1
(43) Pub. Date: Jan. 3, 2019

(54) SYSTEMS AND METHODS FOR FILTERING NOISE AND ANALYZING VENOUS WAVEFORM SIGNALS

(71) Applicants: Baxter International Inc., Deerfield, IL (US); BAXTER HEALTHCARE SA, Glattpark (Opfikon) (CH)

(72) Inventors: Jonathan Handler, Northbrook, IL (US); James Martucci, Libertyville, IL (US); Kyle Hocking, Nashville, TN (US); Susan Eagle, Nashville, TN (US); Colleen Brophy, Nashville, TN (US); Richard Boyer, Somerville, MA (US); Franz Baudenbacher, Nashville, TN (US)

(21) Appl. No.: 16/023,945

(22) Filed: Jun. 29, 2018

Related U.S. Application Data
(60) Provisional application No. 62/527,944, filed on Jun. 30, 2017.
(60) Provisional application No. 62/528,570, filed on Jul. 5, 2017.
(60) Provisional application No. 62/599,421, filed on Dec. 15, 2017.
(60) Provisional application No. 62/671,108, filed on May 14, 2018.

(51) Int. Cl. A61B 5/0215; A61B 5/00; A61M 5/142; A61M 5/172

(52) U.S. Cl. CPC A61B 5/0215; A61B 5/4839; A61B 5/7282; A61M 5/14232; A61M 2230/30; A61B 5/725; A61B 5/7203; A61B 2562/0247; A61M 2205/3331; A61M 5/1723

(57) ABSTRACT

Devices, systems, and methods for filtering medical device noise artifacts from venous waveform signals are disclosed. A peripheral venous pressure (PVP) is measured and transformed from the time domain to the frequency domain for analysis to determine patient status. To avoid artifacts of the pumping, the time-domain PVP measurements are filtered to generate a filtered time-domain PVP signal by removing active pumping periods. The filtered time-domain PVP signal is transformed into a frequency-domain PVP signal, which is analyzed based upon peaks indicating respiratory rate, heart rate, or harmonics thereof. A metric of patient status is then determined from the peaks or corresponding frequencies. The patient status may be related to blood volume of the patient and may be used to control pump operation.

### PRIORITY CLAIM

This application claims priority to U.S. Provisional Application No. 62/671,108, entitled “System and Method for Monitoring and Determining Patient Parameters from Sensed Venous Waveform”, filed May 14, 2018, U.S. Provisional Application No. 62/599,421, entitled “Systems and Methods for Filtering Medical Device Noise Artifacts from Venous Waveform Signals”, filed Dec. 15, 2017, U.S. Provisional Application No. 62/527,944, entitled “System and Method for Filtering Medical Device Noise Artifacts from Venous Waveform Signal”, filed Jun. 30, 2017, and U.S. Provisional Application No. 62/528,570, entitled “System and Method for Utilizing Venous Waveform Signal to Identify and/or Assess Patient Gait, Seizure, Activity or Other Biometrics”, filed Jul. 5, 2017, the entire contents of which are incorporated herein by reference and relied upon.

### BACKGROUND

Proper patient care requires the determination of a plurality of patient status metrics, which are typically measured separately using separate equipment. Measured patient status metrics may be as simple as pulse rate or may be more complex, such as patient body temperature or blood pressure. More complex patient status metrics further include respiratory volume or blood volume. Although various devices and techniques exist to measure various patient status metrics, no comprehensive means of automatically monitoring these various patient metrics exists. Additionally, some important patient characteristics are not typically measured, instead being qualitatively assessed by human observation. Such unmeasured patient characteristics include patient gait, limp, body position, movement, falls, or ambulatory instability. Both using separate measurement devices and relying upon human observation increase system complexity, reduce reliability, and increase cost.

Blood volume metrics are of particular interest because of the complexity of their measurement techniques. Conventional methods of establishing blood volume and related metrics indicative of patient condition have relied upon highly invasive measurements of central venous pressure (herein “CVP”) or other invasive measures, such as Swan-Ganz catheterization. Such invasive measurements require the insertion of a catheter specifically for the purpose of measuring blood pressure within the central portion of the patient's circulatory system. In addition to being highly invasive, the insertion of a catheter solely for the purpose of pressure monitoring increases the complexity of treatment and raises the risk of complications, such as infection. Additionally, CVP measurements may be slower to change in response to certain acute conditions, as the circulatory system attempts to compensate for blood volume disequilibrium (particularly hypovolemia) by protecting blood volume levels in the central circulatory system at the expense of the periphery. For example, constriction in peripheral blood vessels may reduce the effect of fluid loss on the central system, thereby masking blood loss for a period of time in conventional CVP measurements. Such masking can lead to delayed recognition and treatment of patient conditions, resulting in worse patient outcomes.

To address the issues associated with CVP measurements, the use of peripheral intravenous analysis (herein “PIVA”) has been developed, as described in U.S. patent application Ser. No. 14/853,504 (filed Sep. 14, 2015 and published as U.S. Patent Publication No. 2016/0073959) and PCT Application No. PCT/US16/16420 (filed Feb. 3, 2016, and published as WO 2016/126856). Such PIVA techniques measure peripheral venous pressure (herein “PVP”) using intravenous (herein “IV”) lines, such as IV tubing attached to a saline drip or IV pump. In addition to utilizing existing IV lines, the PIVA techniques also include transformation of the PVP measurements into the frequency domain to identify a respiratory rate frequency (F₀) equal to the respiratory rate of the patient and a heart rate frequency (F₁) equal to the heart rate of the patient. Although the PIVA techniques previously disclosed provide an excellent indication of heart rate and blood volume status in certain situations, the disclosure herein further improves upon the previously disclosed PIVA techniques to address challenges related to other situations, improve accuracy, provide earlier warnings of potential problems, or identify additional patient conditions. Similar problems arise in other conventional methods, such as pulmonary artery or capillary pressure measurements.

Monitoring patient metrics during dialysis or other pumping presents a particular challenge to both conventional and PIVA methods. In particular, pumping blood into a patient circulatory system generates a high level of (pressure variation induced) noise related to the pumping cycle. Measured signal values associated with such noise during pumping periods may be orders of magnitude larger than signal values associated with non-pumping periods. Existing techniques for monitoring patient metrics under such conditions involve either shutting down the pump for an extended period or attempting to remove the primary effect of the pump from the measured pressure. Shutting down the pump for extended periods during treatment may be infeasible where consistent pumping is needed, such as during surgery. Even where feasible, such approach can still result in substantial delays in determining the patient status because of the need to interrupt pumping in to obtain measurements. Similarly, existing techniques that attempt to remove the primary effect of the pump address only the principal artifacts introduced by the pump and are sensitive to errors in estimates of the primary effect of the pump. Such techniques also typically require a priori information regarding the operation of the pump (e.g., the amplitude and frequency of pressure waves generated by the pump), and some such techniques further require additional information regarding precise timing of the phases of the pump cycle. Such techniques produce only crude estimates of pressure, which estimates are unsuitable for PIVA or other advanced metrics of patient status. Specifically, such techniques at best remove only approximations of the primary artifacts of pump operation, while leaving numerous secondary artifacts in the measured pressure signal. Moreover, such techniques are dependent upon accurate estimates of the primary pumping artifacts and are sensitive to any errors in the estimates, such as errors caused by variation in pump operation over time. The techniques described herein represent a means of avoiding the respective problems of both types of existing techniques.

Accordingly, systems and methods are needed to filter medical device noise artifacts from venous waveform signals.

### SUMMARY

In light of the disclosure herein, and without limiting the scope of the invention in any way, in a first aspect of the present disclosure, which may be combined with any other aspect listed herein unless specified otherwise, a system for monitoring a patient using a measurement associated with a peripheral venous pressure (PVP) within a peripheral vein of a circulatory system of the patient while the circulatory system of the patient is connected to a pump includes a PVP sensor and an evaluation unit. The PVP sensor includes a transducer disposed adjacent to or connected to an intravenous (IV) tube in fluid connection with the peripheral vein. The PVP sensor is configured to generate an electronic signal associated with the PVP while the circulatory system of the patient is connected to the pump. The evaluation unit includes a computer processor communicatively connected to the PVP sensor to receive the electronic signal and a memory storing non-transitory computer-readable instructions that, when executed by the computer processor, cause the evaluation unit to obtain a time-domain PVP signal comprising values of an electronic signal associated with the PVP from the transducer based upon a physical phenomenon associated with the PVP of the patient over a sample period. The sample period includes a plurality of time segments, including (i) one or more active time segments during which the pump is operating and (ii) one or more inactive time segments during which the pump is not operating. The evaluation unit identifies a first plurality of the values of the time-domain PVP signal associated with the one or more inactive time segments and a second plurality of the values of the time-domain PVP signal associated with the one or more active time segments, based upon evaluation of the values of the time-domain PVP signal. The evaluation unit generates a filtered time-domain PVP signal based upon the first plurality of the values and excluding the second plurality of the values. The evaluation unit applies a transformation to the filtered time-domain PVP signal to generate a frequency-domain PVP signal. The evaluation unit determines a patient status metric for the patient based upon the frequency-domain PVP signal.

In a second aspect of the present disclosure, which may be combined with any other aspect listed herein unless specified otherwise, the pump is a peristaltic IV pump.

In a third aspect of the present disclosure, which may be combined with any other aspect listed herein unless specified otherwise, the pump is configured to operate periodically, such that the one or more active time segments and the one or more inactive time segments periodically alternate.

In a fourth aspect of the present disclosure, which may be combined with any other aspect listed herein unless specified otherwise, the IV tube is disposed between the patient and the pump such that a part of the pump is in fluid connection with the peripheral vein of the circulatory system of the patient via the IV tube.

In a fifth aspect of the present disclosure, which may be combined with any other aspect listed herein unless specified otherwise, the transducer comprises a pressure sensor disposed in fluid connection with an interior of the IV tube, and the physical phenomenon associated with the PVP is a pressure within the interior of the IV tube.

In a sixth aspect of the present disclosure, which may be combined with any other aspect listed herein unless specified otherwise, the executable instructions further cause the evaluation unit to evaluation unit further determine whether the patient status metric indicates a condition of the patient is abnormal, and adjust operation of the pump when the patient status metric indicates the condition of the patient is abnormal by changing a rate of flow of a fluid from the pump into the circulatory system of the patient.

In a seventh aspect of the present disclosure, which may be combined with any other aspect listed herein unless specified otherwise, the executable instructions that cause the evaluation unit to generate the filtered time-domain PVP signal include instructions that cause the evaluation unit to remove the one or more active time segments from the time-domain PVP signal.

In a eighth aspect of the present disclosure, which may be combined with any other aspect listed herein unless specified otherwise, the executable instructions further cause the evaluation unit to generate the filtered time-domain PVP signal by, for each of one or more pairs of the active time segments, identifying one or more corresponding values within both of the active time segments of the pair, and combining the active time segments of the pair by aligning the one or more corresponding values within both of the active time segments of the pair.

In a ninth aspect of the present disclosure, which may be combined with any other aspect listed herein unless specified otherwise, the executable instructions that cause the evaluation unit to generate the filtered time-domain PVP signal include instructions that cause the evaluation unit to estimate a third plurality of values as substitute values for the one or more active time segments, where the third plurality of values are estimated based upon the first plurality of values without reference to the second plurality of values. The executable instructions further cause the evaluation unit to generate the filtered time-domain PVP signal by combining the first plurality of values for the inactive time segments and the third plurality of values for the active time segments.

In a tenth aspect of the present disclosure, which may be combined with any other aspect listed herein unless specified otherwise, the third plurality of values are estimated by performing at least one of regression analysis, forward-backward slope calculation, two-sided slope detection, and mirror matched filtering on at least the first plurality of values.

In a eleventh aspect of the present disclosure, which may be combined with any other aspect listed herein unless specified otherwise, the executable instructions that cause the evaluation unit to determine the patient status metric include instructions that cause the evaluation unit to identify a plurality of frequencies associated with local maxima of the frequency-domain PVP signal, and determine the patient status metric based at least in part upon at least one of the plurality of frequencies associated with the local maxima.

In a twelfth aspect of the present disclosure, which may be combined with any other aspect listed herein unless specified otherwise, the patient status metric is a blood volume metric indicating one or more of the following: hypovolemia, hypervolemia, or euvolemia.

In a thirteenth aspect of the present disclosure, which may be combined with any other aspect listed herein unless specified otherwise, a device for monitoring a patient includes a peripheral venous pressure (PVP) sensor and an evaluation unit. The PVP sensor includes a transducer configured to monitor a physical phenomenon associated with a PVP within a peripheral vein of a circulatory system of the patient while the circulatory system of the patient is connected to a pump. The evaluation unit includes a computer processor communicatively connected to the PVP sensor and a memory storing non-transitory executable instructions that, when executed by the computer processor, cause the evaluation unit to obtain a time-domain PVP signal comprising values of an electronic signal associated with the PVP received from the transducer of the PVP sensor over a sample period. The sample period includes a plurality of time segments, including (i) one or more active time segments during which the pump is operating and (ii) one or more inactive time segments during which the pump is not operating. The evaluation unit identifies a first plurality of the values of the time-domain PVP signal associated with the one or more inactive time segments and a second plurality of the values of the time-domain PVP signal associated with the one or more active time segments, based upon evaluation of the values of the time-domain PVP signal. The evaluation unit generates a filtered time-domain PVP signal based upon the first plurality of the values and excluding the second plurality of the values. The evaluation unit applies a transformation to the filtered time-domain PVP signal to generate a frequency-domain PVP signal. The evaluation unit determines a patient status metric for the patient based upon the frequency-domain PVP signal.

In a fourteenth aspect of the present disclosure, which may be combined with any other aspect listed herein unless specified otherwise, the time-domain PVP signal comprises a first time series of discrete values, the filtered time-domain PVP signal comprises a second time series of discrete values, and the second time series contains at least one segment of a sequential plurality of values within the second time series that are equivalent to a corresponding segment of a sequential plurality of corresponding values within the first time series.

In a fifteenth aspect of the present disclosure, which may be combined with any other aspect listed herein unless specified otherwise, the executable instructions that cause the evaluation unit to generate the filtered time-domain PVP signal include instructions that cause the evaluation unit to remove the one or more active time segments from the time-domain PVP signal.

In a sixteenth aspect of the present disclosure, which may be combined with any other aspect listed herein unless specified otherwise, the executable instructions that cause the evaluation unit to generate the filtered time-domain PVP signal include instructions that cause the evaluation unit to estimate a third plurality of values as substitute values for the one or more active time segments, where the third plurality of values are estimated based upon the first plurality of values without reference to the second plurality of values, and generate the filtered time-domain PVP signal by combining the first plurality of values for the inactive time segments and the third plurality of values for the active time segments.

In a seventeenth aspect of the present disclosure, which may be combined with any other aspect listed herein unless specified otherwise, a method of monitoring a patient using a measurement associated with a peripheral venous pressure (PVP) within a peripheral vein of a circulatory system of the patient while the circulatory system of the patient is connected to a pump includes monitoring, by a transducer, a physical phenomenon associated with the PVP of the patient over a sample period, where the sample period includes a plurality of time segments, including (i) one or more active time segments during which the pump is operating and (ii) one or more inactive time segments during which the pump is not operating. The method includes obtaining, by a processor of an evaluation unit, a time-domain PVP signal comprising values of an electronic signal associated with the PVP from the transducer based upon the monitored physical phenomenon over the sample period. The method includes identifying, by the processor of the evaluation unit, a first plurality of the values of the time-domain PVP signal associated with the one or more inactive time segments and a second plurality of the values of the time-domain PVP signal associated with the one or more active time segments, based upon evaluation of the values of the time-domain PVP signal. The method includes generating, by the processor of the evaluation unit, a filtered time-domain PVP signal based upon the first plurality of the values and excluding the second plurality of the values. The method includes applying, by the processor of the evaluation unit, a transformation to the filtered time-domain PVP signal to generate a frequency-domain PVP signal. The method includes determining, by the processor of the evaluation unit, a patient status metric for the patient based upon the frequency-domain PVP signal.

In a eighteenth aspect of the present disclosure, which may be combined with any other aspect listed herein unless specified otherwise, generating the filtered time-domain PVP signal includes removing the one or more active time segments from the time-domain PVP signal.

In a nineteenth aspect of the present disclosure, which may be combined with any other aspect listed herein unless specified otherwise, generating the filtered time-domain PVP signal includes estimating a third plurality of values as substitute values for the one or more active time segments, where the third plurality of values are estimated based upon the first plurality of values without reference to the second plurality of values, and generating the filtered time-domain PVP signal by combining the first plurality of values for the inactive time segments and the third plurality of values for the active time segments.

In a twentieth aspect of the present disclosure, which may be combined with any other aspect listed herein unless specified otherwise, the third plurality of values are estimated by performing at least one of regression analysis, forward-backward slope calculation, two-sided slope detection, and mirror matched filtering on at least the first plurality of values.

Additional features and advantages of the disclosed devices, systems, and methods are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein. Moreover, it should be noted that the language used in the specification has been principally selected for readability and instructional purposes, and not to limit the scope of the inventive subject matter.

### BRIEF DESCRIPTION OF THE FIGURES

Understanding that the figures depict only typical embodiments of the invention and are not to be considered to be limiting the scope of the present disclosure, the present disclosure is described and explained with additional specificity and detail through the use of the accompanying figures. The figures are listed below.

FIG. 1A illustrates a block diagram of an exemplary PIVA system for use in measuring, analyzing, and responding to a patient's peripheral venous blood pressure, the system having a fluid source.

FIG. 1B illustrates a block diagram of an exemplary PIVA system for use in measuring, analyzing, and responding to a patient's peripheral venous blood pressure, the system not having a fluid source.

FIG. 1C illustrates a block diagram of an exemplary PIVA system for use in measuring, analyzing, and responding to a patient's peripheral venous blood pressure, the system including a sensor disposed within a peripheral vein.

FIG. 1D illustrates a block diagram of an exemplary PIVA system for use in measuring, analyzing, and responding to a patient's peripheral venous blood pressure, the system including a pump.

FIG. 1E illustrates a block diagram of an exemplary PIVA system for use in measuring, analyzing, and responding to a patient's peripheral venous blood pressure, the system including an additional sensor for monitoring patient position or movement.

FIG. 2A illustrates a block diagram of an exemplary PIVA device for implementing some functions of the exemplary PIVA system, showing a fluid connection via a spur of an IV tube.

FIG. 2B illustrates a block diagram of an exemplary PIVA device for implementing some functions of the exemplary PIVA system, showing a fluid connection via a capped IV tube.

FIG. 2C illustrates a block diagram of an exemplary PIVA device for implementing some functions of the exemplary PIVA system, showing a sensor disposed adjacent to an exterior wall of an IV tube.

FIG. 3 illustrates a flow diagram of an exemplary PIVA measurement and analysis method for measuring and analyzing a patient's peripheral venous blood pressure.

FIG. 4A illustrates an exemplary plot of time-domain representation of a PVP signal.

FIG. 4B illustrates an exemplary plot of frequency-domain representation of a PVP signal.

FIG. 5A illustrates an exemplary plot of time-domain representation of a PVP signal during operation of a noise-creating medical device.

FIG. 5B illustrates an exemplary plot of time-domain representation of the PVP signal after removing active time segments during which the medical device is operating.

FIG. 5C illustrates an exemplary plot of time-domain representation of a filtered PVP signal including estimates of values for the removed active time segments.

FIG. 6 illustrates a flow diagram of an exemplary pressure signal filtering method for removing noise artifacts related to operation of a medical device from a signal corresponding to a patient's peripheral venous blood pressure.

FIG. 7 illustrates an exemplary PIVA comparison method for identifying changes in a patient status based upon comparison of PVP over time.

FIG. 8 illustrates a block diagram of exemplary processing performed by an exemplary PIVA module.

FIG. 9 illustrates a block diagram of an exemplary PIVA system, including a PIVA module.

FIG. 10 illustrates a block diagram of exemplary processing performed by an exemplary PIVA module.

FIG. 11 illustrates a flow diagram of an exemplary patient monitoring method using patient PVP.

### DETAILED DESCRIPTION OF EXAMPLE EMBODIMENTS

Although the following text sets forth a detailed description of numerous different embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One of ordinary skill in the art could implement numerous alternate embodiments, which would still fall within the scope of the claims. Unless a term is expressly defined herein using the sentence “As used herein, the term ‘______’ is hereby defined to mean . . . ” or a similar sentence, there is no intent to limit the meaning of that term beyond its plain or ordinary meaning. To the extent that any term is referred to in this patent in a manner consistent with a single meaning, that is done for sake of clarity only, and it is not intended that such claim term be limited to that single meaning. Finally, unless a claim element is defined by reciting the word “means” and a function without the recital of any structure, it is not intended that the scope of any claim element be interpreted based on the application of 35 U.S.C. § 112(f).

In many situations, it is important to monitor various information associated with a patient status or condition. The systems and methods disclosed herein improve upon existing techniques by using metrics or representations of PVP measurements to generate patient status metrics. Such metrics or representations may be generated using frequency-domain PVP data derived from a time-domain PVP signal corresponding to the PVP measurements. Patient status metrics may be generated using a PIVA or other similar system to monitor and respond to changes in a patient's condition, as discussed further herein. The systems, devices, and methods disclosed below enable more efficient and more effective monitoring by using PVP measurements to determine the patient status metrics. This facilitates metric-based monitoring for a broader range of patient conditions that were previously susceptible to automatic monitoring. This also facilitates monitoring of distinct types of patient conditions based upon measurements indicative of pressure in a peripheral vein, without needing specialized sensors to monitor each type of patient condition. Exemplary embodiments are described below.

### PIVA System and Signal Noise

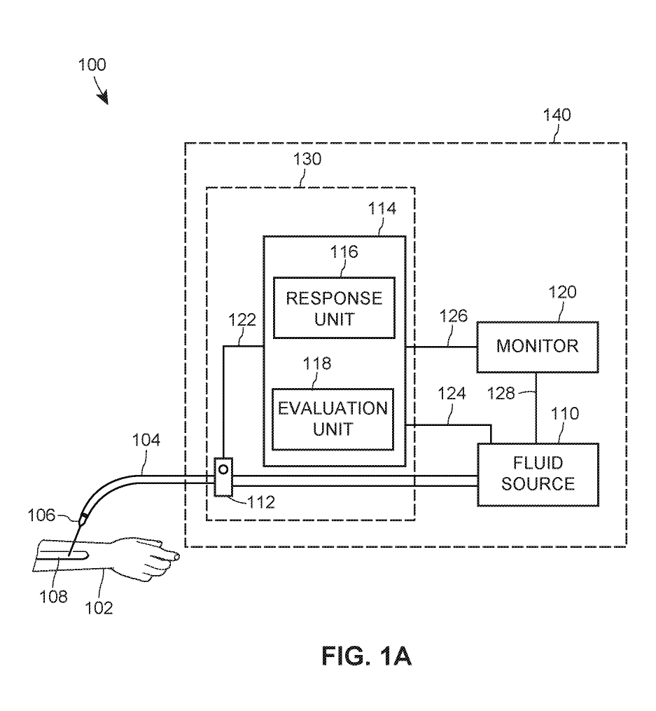

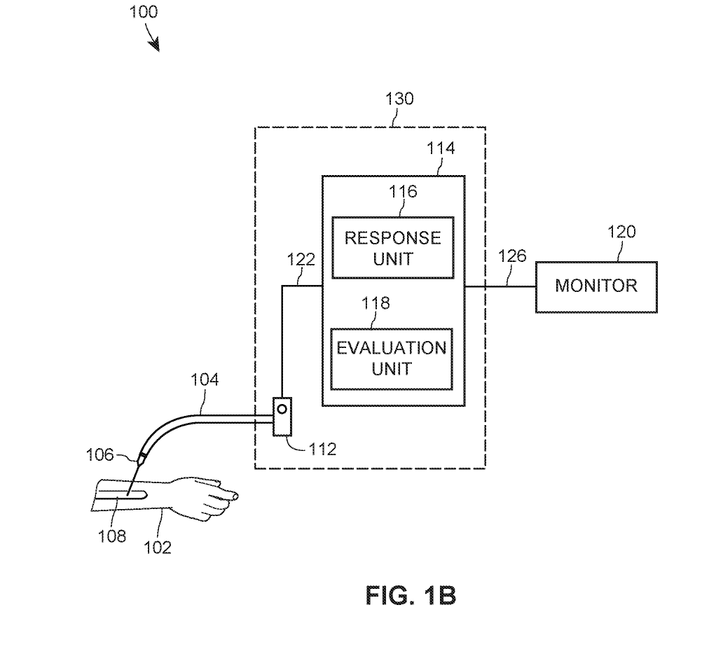

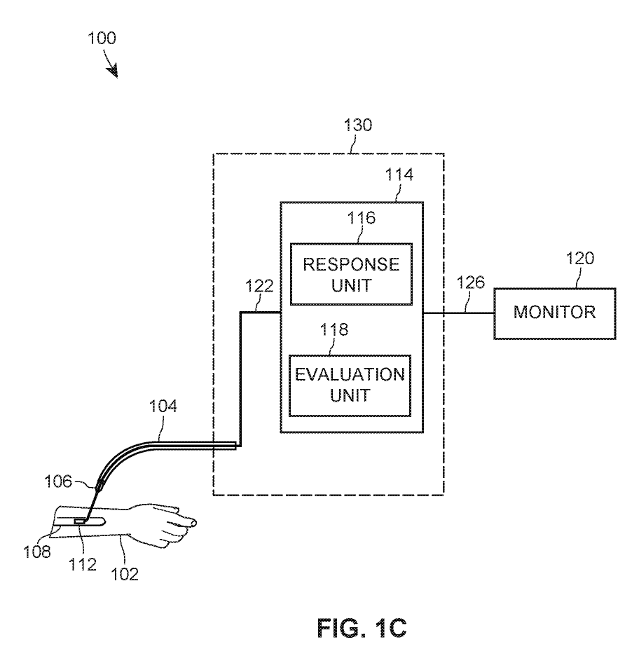

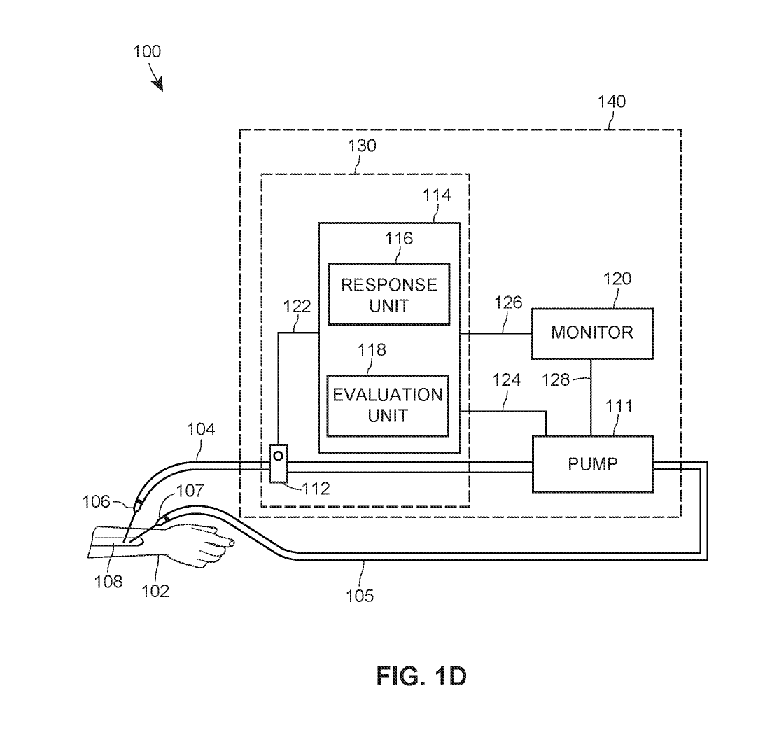

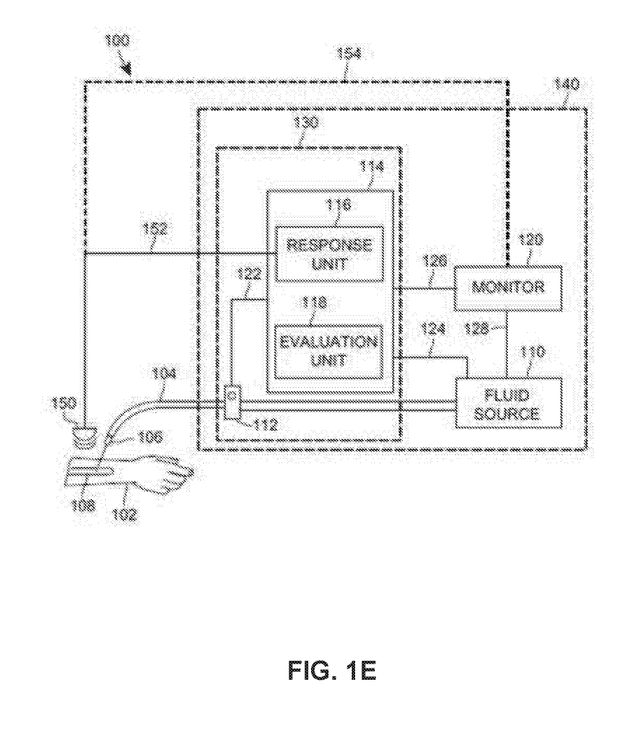

FIGS. 1A-E illustrate block diagrams of embodiments of an exemplary PIVA system 100 for use in measuring, analyzing, and responding to peripheral venous blood pressure of a patient 102. The exemplary PIVA system 100 or a similar system may be used to implement the various techniques for monitoring patient status based upon measurements associated with PVP for the patient 102. The PIVA system 100 may measure a pressure signal associated with the patient's peripheral vein, analyze the pressure using PIVA techniques to identify key frequency components of the pressure signal, and analyze the key frequency components of the pressure signal to determine patient status based upon one or more metrics, as discussed below.

The exemplary PIVA system 100 illustrated in FIG. 1A includes an IV tube 104 in fluid connection with the circulatory system of the patient 102. Specifically, a venous access device 106 may be inserted into a peripheral vein 108 of the patient 102 at an access point. The venous access device 106 may include a needle, catheter, cannula, or other means of establishing a fluid connection between the IV tube 104 and the peripheral vein 108. The venous access device 106 may be a separate component connected to the IV tube 104 or may be formed as an integral portion of the of the IV tube 104. In either case, the venous access device 106 may include a terminal end inserted into the peripheral vein 108 at the access point and a connecting end that connects to a primary portion of the IV tube 104. The primary portion of the IV tube 104 may serve as a conduit between the venous access device 106 and a fluid source 110.

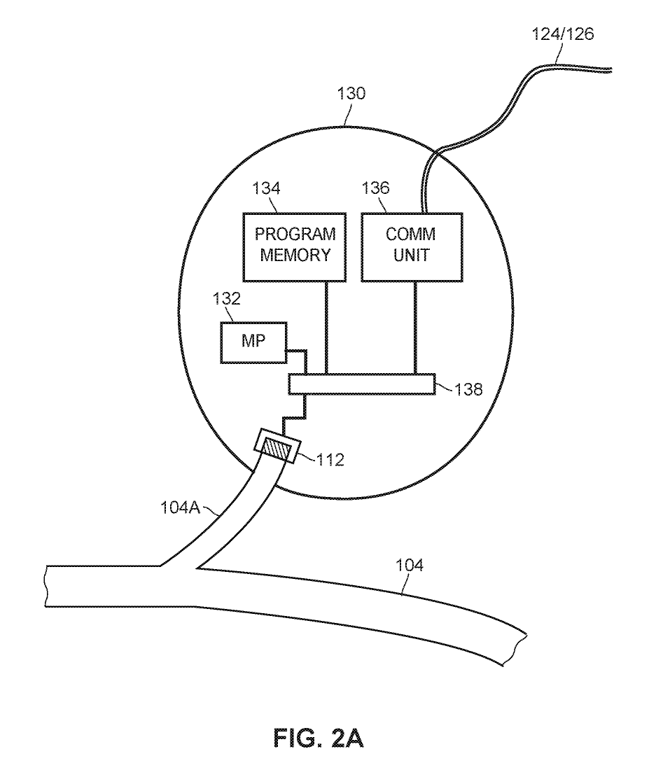

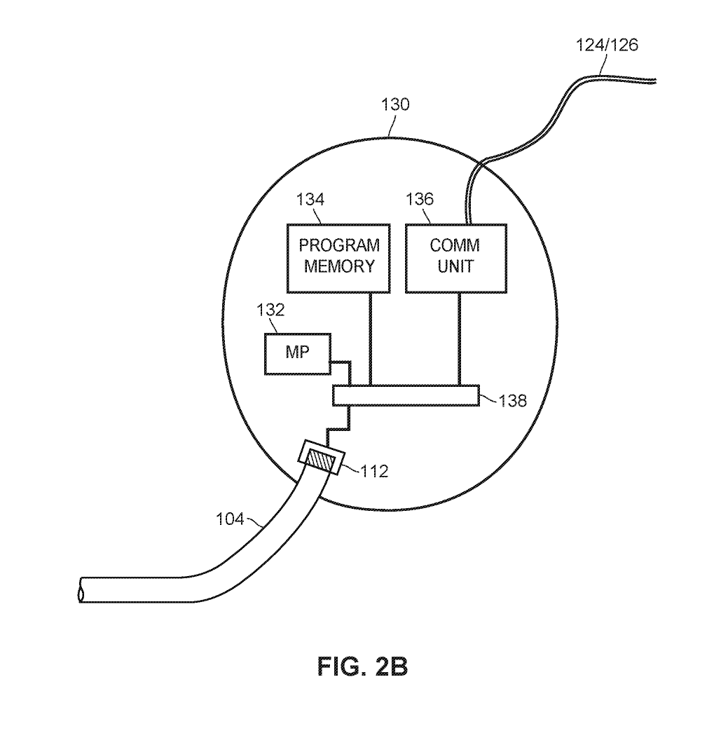

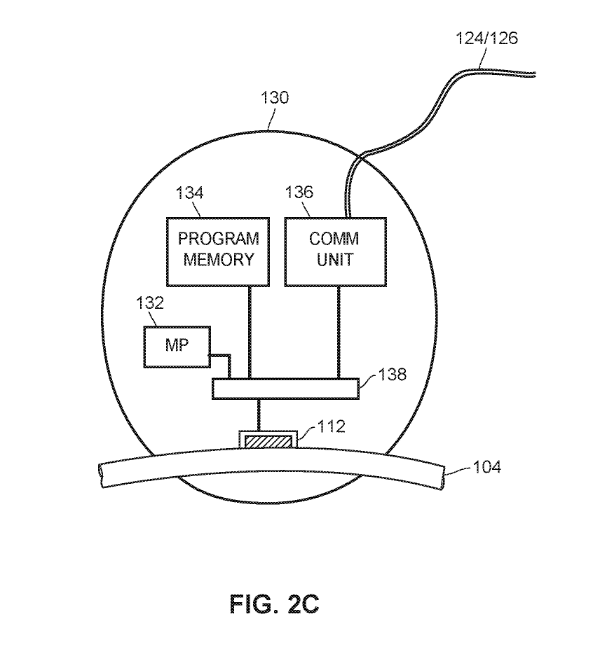

At some point along the primary portion of the IV tube 104, a pressure sensor 112 may be disposed to monitor a physical phenomenon associated with PVP of the patient 102. In some embodiments, the pressure sensor 112 may directly measure a pressure corresponding to the PVP, such as a pressure in the interior of the IV tube 104. In such embodiments, a measuring portion of a pressure transducer (e.g., a Piezoelectric pressure transducer) may be disposed in fluid connection with the interior of the IV tube 104. The pressure sensor 112 may thus also be in fluid connection with the peripheral vein 108 of the patient through the IV tube 104 and the venous access device 106. The pressure sensor 112 is thereby enabled to measure pressure changes in the peripheral venous system of the patient 102 based upon changes in the fluid pressure within the IV tube 104. In other embodiments, the pressure sensor 112 may indirectly measure a pressure corresponding to the PVP of the patient 102 by measuring other phenomena, without being disposed in fluid connection with the interior of the IV tube 104. For example, the pressure sensor 112 may instead be attached to the exterior of the IV tube 104 and thereby disconnected from the interior of the IV tube 104 or the fluid of the fluid source 110 (as illustrated in FIG. 2C). The pressure sensor 112 may, in some such embodiments, measure pressure based upon acoustic or optical phenomenon at the sensor location. In some embodiments, the pressure sensor 112 may be disposed at a terminating end (i.e., a capped off end) of an IV tube 104 inserted specifically for the purpose of measuring pressure within the peripheral vein 108, in a manner similar to that illustrated in FIG. 1B. In further embodiments, other sensors may be used instead of the pressure sensor 112, such as sonic, electrical, temperature, or similar sensors to measure one or more of the following physical phenomena: pressure, sound, electrical resistivity or conductivity, electrical voltage or current, light levels or properties (e.g., spectrum or frequency shifts), or other similar phenomena. Whichever types of sensors are used, the sensors may be (but need not be) in fluid contact with the peripheral vein 108 of the patient through the IV tube 104 and the venous access device 106 (or directly through the venous access device 106) to measure the phenomena associated with the PVP of the patient 102. In yet further embodiments, the sensor 112 may be disposed within a portion of a needle, catheter, or other venous access device 106 that is inserted within the peripheral vein 108 of the patient 106, as illustrated in FIG. 1C. Thus, the PVP may be measured in situ within the peripheral vein 108. Such in situ measurement is advantageous inasmuch as it obviates the effect of temperature, viscosity, and other factors on transmission of pressure within the IV tube 104.

In various embodiments, the pressure sensor 112 may be positioned at various distances from the access point of the peripheral vein 108, from a location within the peripheral vein 108 or a location proximate to the connecting end of the venous access device 106 to a position proximate to the fluid source 110 or at a terminating end of the IV tube 104. The pressure sensor 112 is illustrated in FIG. 1A as being at an intermediate location along the length of the IV tube 104 in order to illustrate better the various components of the PIVA system 100. In some embodiments, the pressure sensor 112 may directly measure fluid pressure within the IV tube 104. Specifically, the pressure sensor 112 may include a transducer that provides an electronic pressure signal indicative of the pressure detected by the transducer to an analysis component 114 via a connection 122. The electronic pressure signal may be an analog electrical signal directly provided by the transducer or may be a preprocessed digital signal indicating pressure values based upon the transducer interface with the primary portion of the IV tube 104. In embodiments in which the pressure sensor 112 is not in fluid connection with the IV tube 104 or the peripheral vein 108, the pressure sensor 112 may nonetheless include one or more transducers to generate electronic signals associated with the PVP. For example, the pressure sensor 112 may use one or more microphones disposed to detect sound at an exterior surface of an IV tube 104 to generate electronic pressure signals indicative of pressure within the IV tube 104 as a proxy for PVP within the peripheral vein 108.

The analysis component 114 is communicatively connected to the pressure sensor 112 to receive the electronic pressure signal via the connection 122. The analysis component 114 may include general-purpose or special-purpose processing hardware, such as microprocessors or special-purpose analysis circuits. As shown, the analysis component 114 may include one or more units for performing the PIVA analysis. A response unit 116 may identify and control responses based upon the pressure data from the pressure sensor 112. The response unit 116 may control the presentation of alarms or may control the operation of the fluid source 110, such as by controlling the rate of fluid flow. To determine appropriate responses, the response unit 116 may receive evaluation data from an evaluation unit 118, which may include metrics determined from the electronic pressure signal. The evaluation unit 118 may obtain pressure values (or signal values directly or indirectly associated with PVP) from the electronic pressure signal and evaluate the pressure values to determine information regarding the patient 102, such as blood volume metrics, position metrics, movement metrics, or other metrics as described in further detail below. The information generated by the evaluation unit 118 may also be stored or presented for patient monitoring. In alternative embodiments, additional, fewer, or alternative units may be included. For example, the evaluation unit 118 may perform the functions ascribed to the response unit 116 herein.

The analysis component 114 may be communicatively connected to a monitor 120 via a connection 126 in some embodiments. The monitor 120 may be a separate monitor for displaying information regarding the patient or may be incorporated into another device, such as a pump or other fluid source device. The monitor 120 may also be communicatively connected to the fluid source 110 via a connection 128 to receive and display information associated with the fluid source 110. In some embodiments, the monitor 120 may be used to control the operation of the fluid source 110, such as by adjusting fluid flow rate, duration of operation, mode of operation, or other similar control. The analysis component 114 may similarly be communicatively connected to the fluid source 110 via connection 124 in some embodiments. The analysis component 114 may receive information regarding operation of the fluid source 110 for use in evaluating the patient by the evaluation unit 118. The response unit 116 may also communicate with the fluid source 110 to control operation of the fluid source 110 in response to information regarding the patient determined based upon the electronic pressure signal from the pressure sensor 112.

In some embodiments, the fluid source 110 may comprise a pump 111, as illustrated in FIG. 1D. Such pump may be disposed within the exemplary PIVA system 100 to pump blood or other fluids into the peripheral vein 108 of the patient 102. For example, the pump 111 may include an IV infusion pump or a dialysis pump, such as a peristaltic pump. The pump 111 may be configured to operate cyclically in a periodic or aperiodic manner, having alternating intervals of operation (i.e., active time segments) and rest (i.e., inactive time segments). By alternating the pump 111 between operating and rest intervals, periods of time in which the pump 111 is not operating may be used for PIVA analysis, as described further below. In some embodiments, such as where the pump 111 is a hemodialysis pump, the pump 111 may further be connected to the circulatory system of the patient 102 by an additional IV tube 105 (which may include or be further attached to an additional venous access device 107), thereby creating an extracorporeal blood circuit through the pump 111 via the tubes 104 and 105. In such embodiments, the pump 111 may draw blood out of the patient 102 through either of tubes 104 or 105. The extracorporeal blood may then be processed according to a therapeutic regimen before being returned to the patient circulatory system (or may be replaced by another fluid that may be infused into the patient circulatory system) through the other of the IV tubes 105 or 104. Although described herein as one component, it should be understood that the pump 111 may comprise a plurality of pumping components (e.g., a pair of pumps for extracting and returning blood or other fluids, or multiple pumps in a common fluid system) in some embodiments.

In some embodiments, the exemplary PIVA system 100 may include one or more additional sensors 150, as illustrated in FIG. 1E. The additional sensors 150 may include pressure sensors, infrared sensors, optical sensors, magnetic sensors, or the like. In various embodiments, each additional sensor 150 may be connected to the analysis component 114 via a connection 152 or to the monitor 120 via connection 154, which may be wired or wireless connections. Such additional sensors 150 may be disposed to monitor the presence, absence, location, or position of the patient 102. For example, a pressure sensor may be disposed within a hospital bed to determine whether the patient 102 is within the bed based upon a measurement of weight. Similarly, one or more sensors may be disposed to determine whether such bed is flat or is partially elevated to facilitate a sitting posture. Other additional sensors 150 may be disposed upon the patient 102 to monitor movement. For example, a wristband sensor containing an accelerometer array may be worn by the patient 102, which may measure data regarding at least some patient movements. The additional sensors 150 may thus be disposed together with the pressure sensor 112 within a PIVA device 130 or may be separate therefrom. In further embodiments, the additional sensors 150 may further include any of the following to measure orientation or motion of the patient: a real-time three-dimensional gyroscope, one or more cameras monitoring the local physical environment around the patient, or a microphone configured to monitor sounds in the local physical environment. Sensor data from the additional sensors 150 may be correlated with IV pressure measurements or other pressure-related measurements associated with the PVP of the patient.

The various connections 122, 124, 126, and 128 may each be wired or wireless connections in various embodiments. Moreover, some or all of the connections 122, 124, 126, and 128 may be internal to devices, such as a PIVA device 130 or a PIVA-integrated fluid source 140.

The PIVA device 130 may incorporate the pressure sensor 112 and analysis component 114 (along with associated connections) into a device that may be attached to or within the IV tube 104 to perform PIVA monitoring of the patient 102. In some embodiments, the PIVA device 130 may further include one or more additional sensors 150 or other components described herein. The PIVA-integrated fluid source 140 may include a computer-controlled fluid reservoir or pump configured to utilize PIVA monitoring of the patient 102 in controlling fluid flow. Like the PIVA device 130, the PIVA-integrated fluid source 140 may include the pressure sensor 112 and analysis component 114, along with the fluid source 110 and the monitor 120 (along with associated connections). Alternative embodiments may include additional, fewer, or alternative components in alternative configurations.

FIGS. 2A-C illustrate block diagrams of exemplary embodiments of a PIVA device 130 for implementing some functions of the exemplary PIVA system 100. As illustrated in FIG. 2A, the exemplary PIVA device 130 may be configured to attach to a spur 104A of the IV tube 104, such as at one branch of a Y-connector or a T-connector. Alternatively, the exemplary PIVA device 130 may be configured to attach to a terminal end of the IV tube 104, as illustrated in FIG. 2B. In such embodiments, the PIVA device 130 may cap a terminating portion of the IV tube 104, such that no fluid source 110 is connected to the peripheral vein 108 through the same IV tube 104. Of course, a fluid source could be otherwise connected to provide fluids to the patient 102 via another IV tube and another venous access device. In further embodiments, the PIVA device 130 may be configured to attach to the exterior of the IV tube 104, as illustrated in FIG. 2C. In such embodiments, one or more sensors of the PIVA device 130 may monitor PVP without being in fluid connection with the peripheral vein 106 or the interior of the IV tube 104.

As discussed above, the PIVA device 130 may include a pressure sensor 112 disposed such that a sensing portion is in contact with fluid in the IV tube 104, as illustrated in FIGS. 2A-B. In some embodiments, the pressure sensor 112 (or an alternative sensor) may instead be external to the IV tube 104, as illustrated in FIG. 2C. However situated, the pressure sensor 112 is disposed to monitor a physical phenomenon associated with pressure in the peripheral vein 108. Such physical phenomenon may include pressure in the IV tube 104, expansion or contraction of the IV tube 104, sound in the IV tube 104, vibrations of the IV tube 104, or other similar phenomena. The pressure sensor 112 may be electrically communicatively connected to a microprocessor 132 via a system bus 138. The microprocessor 132 (MP) may be further communicatively connected to a program memory 134 and a communication unit 136 (COMM UNIT) via the system bus 138. The program memory 134 may be a non-transitory, non-volatile memory (e.g., a flash memory) storing executable instructions that may be executed by the microprocessor 132 to evaluate the electronic pressure signal from the pressure sensor 112, determine patient information (e.g., blood volume metrics), determine appropriate responses to the determined patient information, and control the communication unit 136 to electronically communicate with the fluid source 110 or monitor 120 via connections 124 or 126. The program memory 134 may store a plurality of routines, scripts, or modules corresponding to units or sub-units of the analysis component 114, such as software modules corresponding to response unit 116 or the evaluation unit 118.

The communication unit 136 may be a hardware component configured to send and receive electronic data between the PIVA device 130 and the fluid source 110 or monitor 120 via connections 124 or 126. The connections 124 and 126 are illustrated as being wired connections in the exemplary PIVA device 130, which may also be used to obtain power for the PIVA device 130. Alternatively, another power connection or battery (not shown) may provide power to the PIVA device 130. Although shown as separate wired connections, the connections 124 and 126 may be separate or combined wired or wireless connections. The connections 124 and 126 may communicate with a communication component of the fluid source 110 or monitor 120, which may include or be part of a pump 111. Such communications may include raw data generated by the pressure sensor 112, processed data related to measurements by the pressure sensor 112, data analyzed according to the methods described below, or alert signals or control commands determined based upon analyzed data. The fluid source 110 or monitor 120 may then take appropriate action or present appropriate information based upon the communications from the exemplary PIVA device 130.

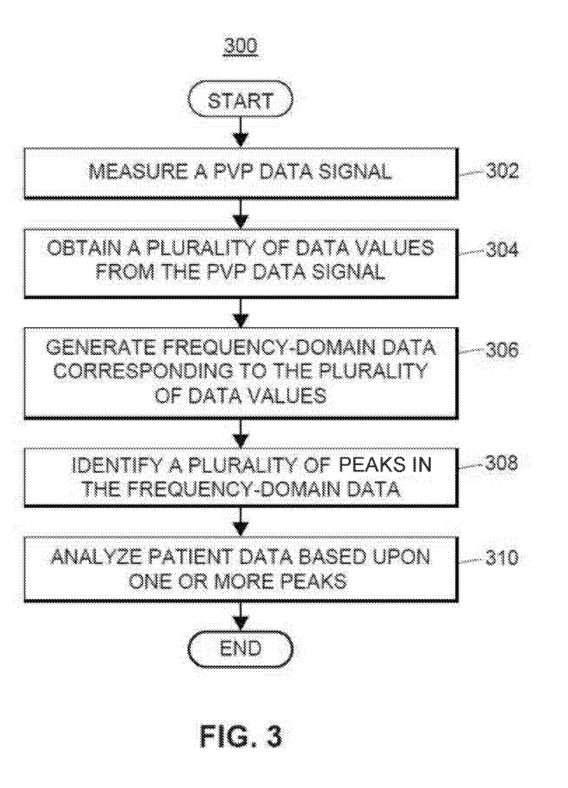

FIG. 3 illustrates a flow diagram of an exemplary PIVA measurement and analysis method 300 for measuring and analyzing a status of a patient 102 based on PVP using the PIVA system 100. The method 300 may be used to determine various patient status metrics, such as metrics related to patient blood pressure, blood volume, respiration, position or movement, or systemic vascular resistance. The method 300 may be performed by the evaluation unit 118 using an electronic pressure signal from the pressure sensor 112, the generation of which electronic pressure signal by the pressure sensor 112 may be included in the method 300 in some embodiments.

The method 300 begins with measuring a PVP data signal for the patient 102 (block 302). The PVP data signal may be measured by using a transducer of the pressure sensor 112 to generate an electronic pressure signal indicating PVP based upon a physical phenomenon associated with PVP. For example, this may be accomplished by measuring the pressure within the IV tube 104. Because the IV tube 104 is in fluid connection with the peripheral vein 108 of the patient 102 via the venous access device 106, the pressure in the IV tube 104 measured by the pressure sensor 112 is associated with patient PVP (i.e., the pressure in the peripheral vein 108). In some embodiments of the PIVA system 100, the pressure within the IV tube 104 may be different from the PVP within the peripheral vein 108, but the pressure measured within the IV tube 104 may nonetheless be proportional to the PVP in the peripheral vein 108. Thus, the measured PVP data signal may be adjusted to compensate for differences between the pressures, if desired. For example, adjustments may be made based upon temperature, viscosity of the patient's blood or a fluid provided by the fluid source 110, or a gauge or rigidity of the IV tube 104. Whether adjusted or unadjusted, the PVP data signal measured by the pressure sensor 112 accurately represents changes in pressure over time, including both periodic pressure changes associated with respiratory and circulatory cycles and aperiodic pressure changes that may be indicative of changes in patient condition. Similarly, a PVP data signal generated by the pressure sensor 112 by components not in fluid contact with the interior of the IV tube 104 likewise provides a representation of the pressure within the peripheral vein 108 of the patient 102. The PVP data signal may be the electronic pressure signal generated by the pressure sensor 112 or may be a data signal derived therefrom. In alternative embodiments, the PVP data signal may be evaluated in real-time as it is generated, or it may be stored for later analysis. Depending upon the components used to measure the PVP-related phenomenon, the PVP data signal may be generated or stored as an analog (i.e., as a continuous function or curve over a time segment) or a digital signal (i.e., as a set of discrete values representing distinct times).

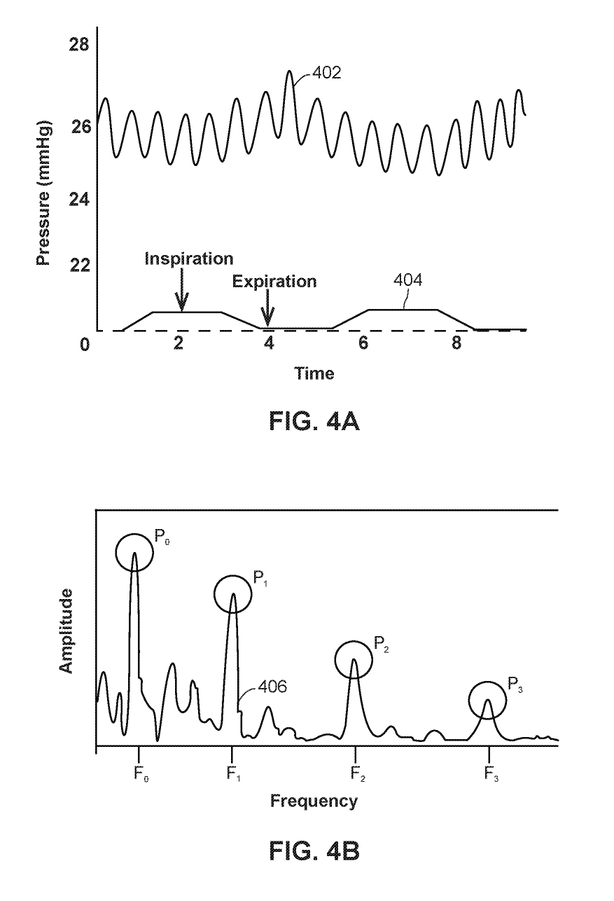

FIG. 4A illustrates an exemplary chart of a time-domain representation of the PVP data signal, which may be the electronic pressure signal from the pressure sensor 112. The chart illustrates a time-domain PVP signal 402, which shows periodic increases and decreases in pressure associated with the patient heartbeat. Additionally, the time-domain PVP signal 402 exhibits slower cyclical variation as a result of patient respiration. The chart also illustrates a respiration curve 404 that shows the effect of inspiration and expiration on the time-domain PVP signal 402. Because of the expansion of the lungs during inspiration, the measured pressure in the peripheral vein is higher during inspiration than during expiration, when the volume of the lungs is reduced. Other factors influence PVP, such as blood volume and patient movement.

The time-domain PVP signal 402 is thus a combination of a plurality of influences, both periodic (e.g., heart rate or respiration) and aperiodic (e.g., movement or blood loss). Because the resulting time-domain PVP signal 402 will include noise from various sources, it may be difficult to detect small changes in pressure that may serve as indications of patient status. Therefore, PIVA techniques utilize a frequency-domain evaluation of the PVP data signal in some embodiments, as described below. In other embodiments, time-domain or mixed techniques may also be used to evaluate patient status or generate patient status metrics. It should be recognized that, although the time-domain representation of the PVP data signal is illustrated graphically as a chart in FIG. 4A to illustrate the salient features of the data, it is not necessary to produce a chart or other graphical representation of such data signal. Instead, in some embodiments, the PVP data signal is processed by the evaluation unit 118 without generating a graphical representation of the time-domain PVP data signal, or the graphical representation may be generated for user review separately from evaluation.

Returning to FIG. 3, a plurality of data values may then be obtained from the measured PVP data signal (block 304). The evaluation unit 118 may sample values of the live or stored PVP data signal to obtain the plurality of data values. In some embodiments, the data values may be sampled at fixed intervals over a period of time to obtain a plurality of data values within an evaluation window, which may include storing the plurality of data values associated with the window in temporary or permanent electronic data storage. In further embodiments, data for multiple evaluation windows may be obtained, such that each evaluation window includes a plurality of data values. For example, concurrent time periods may be identified as separate evaluation windows, or evaluation windows may be identified as time periods separated by an intervening period (e.g., twenty-second evaluation windows beginning every minute, thus separated by forty-second intervening periods). When the evaluation unit 118 samples values of a live (continuously updating) PVP data signal, in some embodiments, the evaluation window may be updated on a rolling basis to obtain new data values while covering time periods of fixed duration. For example, the evaluation window may be repeatedly updated by adding new sample data values and removing the oldest sample data values to maintain a window of a fixed duration (e.g., five seconds, ten seconds, twenty seconds, or some other time period) of the most recent PVP data from the pressure sensor 112. Where the evaluation unit 118 periodically obtains updates of new sample data values, the window may be updated (and the transformation and evaluation described below may be performed for the updated window) every time a new data value is received. In an alternative embodiment, the plurality of data values may correspond to the continuous values of an analog PVP data signal, which may be obtained and analyzed by analog electronic equipment (which may be part of the evaluation unit 118).

From the plurality of data values, the evaluation unit 118 generates frequency-domain data corresponding to the plurality of data values (block 306). Such frequency-domain data may be generated as a frequency distribution representing the PVP data signal in the frequency domain as magnitudes associated with each of a plurality of frequencies. This may include applying a data transformation to the plurality of data values representing a time-domain PVP signal to produce a frequency-domain representation of the PVP signal. In a preferred embodiment, the evaluation unit 118 applies a fast Fourier transform (FFT) to the sampled plurality of data values to generate a frequency-domain representation of the PVP signal. In a different embodiment, a different data transform (e.g., Laplace transform, Mellin transform, Hartley transform, short-time Fourier transform, Chirplet transform, Hankel transform, or any other continuous or discrete transform) may be implemented to transform data to a frequency-domain representation of the PVP signal. The FFT may be applied periodically (e.g., every ten seconds, every minute, or every two seconds, with or without overlapping evaluation windows). In some embodiments, other analysis techniques that can identify local maxima according to frequency are contemplated, such as wavelet transform, autocorrelation, or other signal analysis techniques that can segregate contributions to signal spectral energy content over time-domain segments.

The frequency-domain data may include a plurality of values representing the magnitude of various frequency components in the measured PVP data signal based upon the plurality of data values. Such values may be discrete or may be part of a curve of magnitudes corresponding to frequencies, which curve may be generated by interpolation or approximation between a finite number of values associated with a finite number of frequencies. Although FFT algorithms may be used to great effect, other time-frequency transforms or other techniques of analyzing frequency components of signals may be utilized to evaluate the plurality of data values. For example, in addition to other Fourier transforms, the evaluation may include wavelet transforms or time-frequency representations of the measured PVP data signal.

FIG. 4B illustrates an exemplary chart of a frequency-domain representation of the PVP data signal, corresponding to the time-domain PVP signal 402 represented in the time domain in FIG. 4A. The chart illustrates the magnitude of each frequency component by a frequency curve 406. As is customary, the horizontal axis represents frequency, and the vertical axis represents magnitude. Although the chart is exemplary, certain typical features may be discerned therein. Of particular interest are the several peaks (P_{N}) of the frequency curve 406 associated with frequencies (F_{N}). Between the peaks, minor variations in magnitude are seen, which may represent minor components of the time-domain PVP signal 402 associated with noise in the system or artifacts of the circulatory system of the patient 102 (e.g., movements of the patient during measurement, or openings and closings of the atrioventricular and aortic valves) or in the exemplary PIVA system 100 (e.g., pump noise).

Although the frequency-domain representation of the PVP data signal is illustrated in FIG. 4B as a chart to illustrate the salient features, it should be understood that it is not necessary to produce a chart or other graphical representation of the frequency-domain data. Indeed, in some embodiments, no such graphical representation is generated. Instead, the frequency-domain data is processed by the evaluation unit 118 as an intermediate process, the results of which are not directly presented to a user of the system or device. In some embodiments, the frequency-domain data may be stored in transitory or non-transitory memory as values within a data list, data table, or similar data structure.

Under ordinary conditions, the peak (P₀) with the lowest frequency (F₀) corresponds to the respiration rate of the patient 102, and the peak (P₁) with the next-lowest frequency (F₁) corresponds to the heart rate of the patient 102. One or more harmonic peaks (P_{H}) associated with harmonic frequencies (F_{H}) of the heart rate frequency (F₁) may be identified in some embodiments. Such harmonic peaks (P_{H}) are associated with local maxima of the frequency curve 406. The next two peaks (P₂) and (P₃) of the frequency curve 406 are harmonic peaks (P_{H}) occurring at frequencies associated with the first and second harmonics of the heart rate at the first harmonic frequency (F₂) and the second harmonic frequency (F₃). The harmonics occur at fixed multiples of the heart rate frequency (F₁). Typically, these multiples are typically integer multiples. Specifically, experimental data indicate that first harmonic frequency (F₂) is approximately twice the heart rate frequency (F₁), and the second harmonic frequency (F₃) is approximately thrice the heart rate frequency (F₁).

Identification of the peaks (e.g., P₁, P₂, P₃) of the corresponding frequencies (e.g., F₁, F₂, F₃), such as via the evaluation unit 118, provides for subsequent calculations of patient status (e.g., hemodynamic status). For example, the peaks (e.g., P₁, P₂, P₃) of the corresponding frequencies (e.g., F₁, F₂, F₃) may be used to calculate a PIVA Score, as further detailed herein.

Although not shown, additional peaks associated with third and higher harmonics of the heart rate may be identified in some embodiments. The further harmonic frequencies (F₄, F₅, . . . F_{N}) typically occur at corresponding sequential integer multiples of the heart rate frequency (F₁). For example, a second harmonic frequency may be represented by F₃, a third harmonic frequency may be represented by F₄, etc. Although some variation exists in the observed frequencies of the peaks associated with the harmonic frequencies, the harmonic frequency peaks have been found to occur at frequencies that are typically within a range of approximately ten percent (i.e., ±10%) of the value of the heart rate frequency above or below the integer multiples of the heart rate frequency (F₁). The relationships between the magnitudes of the peaks (P_{N}) may vary, but the magnitude of the peak (P₁) associated with the heart rate frequency (F₁) should be greater than the magnitudes of the peaks (P₂), (P₃), etc., associated with the harmonic frequencies (F₂), (F₃), etc., thereof.

Furthermore, it should be noted that while FIG. 4B illustrates the frequency curve 406 as a number of parabolic peaks (e.g., P₀, P₁, P₂, P₃), other graphical representations of the frequency-domain representation should be expected. For example, to the extent that the system is consistent (e.g., consistent patient respiration and heart rate), and the sampling rate is high enough (e.g., the sampling rate of data values measured in the time domain), the peaks (e.g., P₀, P₁, P₂, P₃) may be depicted graphically as vertical lines (e.g., parabolic peaks with unperceivable width or parabolic peaks with no width).

Although the present disclosure generally refers to the respiration rate as corresponding to the lowest-frequency peak (P₀), the heart rate as corresponding to the next-lowest frequency peak (P₁), and so on, it should be appreciated that any such reference is done for ease of explanation. To this end, in some embodiments, the time-domain PVP signal may detect one or more frequencies lower than the respiration rate. For instance, gut frequencies tend to be associated with lower frequencies than a typical respiratory frequency. In these embodiments, the peak (P₀) with the lowest frequency (F₀) corresponds to a gut frequency, and the peak (P₁) with the second-lowest frequency (F₁) corresponds to the respiratory frequency. Similarly, the heart rate frequency and each of the corresponding harmonic frequencies would correspond to the next-lowest peak (P₂) and the following peaks (P₃, P₄, . . . P_{N}), respectively. It should be appreciated that in some further embodiments, the time-domain PVP signal may detect multiple frequencies lower than the respiratory frequency. Accordingly, the peak index corresponding to the respiration rate, the heart rate, and the heart rate harmonics may increase by the number of frequencies detected lower than the respiration rate. As such, unless specifically described otherwise, any reference to the respiratory rate corresponding to the lowest frequency peak (P₀) and the heart rate frequency corresponding to the next-lowest frequency peak (P₁) is not limiting and also envisions offsetting the correspond peak indexes by the number of lower-than-respiration rate frequencies detected by the time-domain PVP signal.

Turning again to FIG. 3, the evaluation unit 118 further identifies a plurality of frequencies (F_{N}) corresponding to peaks (P_{N}) of the frequency-domain representation of the PVP signal (block 308), such as the frequency curve 406. The evaluation unit 118 may first identify values indicating peaks (P_{N}) in the frequency-domain representation of the PVP signal by comparison of the frequency-domain PVP signal values, then identify the corresponding frequencies (F_{N}) associated with the identified peak values (P_{N}). To determine the peak values (P_{N}), the evaluation unit 118 may utilize any of various methods to identify local maxima as peaks, including methods based upon any or all of a comparison of the relative magnitudes of local maxima, establishment of fixed or dynamic frequency bands around each peak, or comparison of full width at half maximum for local maxima. For example, a band-pass filter may be employed to separate segments of the frequency-domain representation of the PVP signal to further identify local maxima. This may be particularly useful in identifying harmonic peaks (P_{N}) and corresponding harmonic frequencies (F_{H}) because such harmonics occur at integer multiples of the heart rate frequency (F₁).

As an example, a band-pass filter centered around a frequency twice the heart rate frequency (F₁) and having a band width of twenty percent of the heart rate frequency (F₁) may be used to define a range of the frequency-domain representation of the PVP signal that contains the first harmonic peak (P₂). The first harmonic frequency (F₂) may then be identified by simply determining the frequency associated with the local maximum value of the frequency-domain representation of the PVP signal within such range. By employing these or other known techniques, the peaks (P_{N}) of the frequency-domain representation of the PVP signal may be distinguished from other local maxima arising from noise or other minor phenomena in the circulatory system.

Once the plurality of frequencies (F_{N}) associated with the peaks (P_{N}) have been identified, the evaluation unit 118 may analyze the magnitudes of the frequency-domain representation of the PVP signal at one or more of the frequencies (F_{N}) to determine one or more aspects of patient status (block 310). Such analysis may include determining one or more patient status metrics, such as a blood volume metric, respiratory volume metric, patient position metric, patient movement metric, systemic vascular resistance metric, other metric relating to the systemic vascular resistance (e.g., mean arterial pressure, mean venous pressure, cardiac output), or the like for the patient 102. For example, the patient status metrics may include a blood volume metric indicating one of the following hemodynamic states of the patient 102: hypovolemia, hypervolemia, or euvolemia. Hemodynamic states of the patient 102 may be determined as a score or as a category of patient status in various embodiments. In further embodiments, time-domain analysis may additionally or alternatively be performed to evaluate the PVP signal, as discussed elsewhere herein.

Some patient status metrics may be determined directly from the one or more frequencies (F_{N}) or magnitudes of the frequency-domain representation of the PVP signal associated therewith. For example, respiratory depth may be determined based upon the magnitude associated with the respiratory frequency (F₀) (i.e., the magnitude of the respiratory peak (P₀)), or a blood volume metric may be determined based upon the magnitude associated with the heart rate frequency (F₁) (i.e., the magnitude of the heart rate peak (P₁)). As another example, a blood volume metric indicative of patient hemodynamic state (e.g., hypovolemia or hypervolemia) may be directly measured or calculated.

For example, as previously mentioned, subsequent to performing transformation, the evaluation unit 118 may identify the peaks (e.g., P₁, P₂, P₃) of the corresponding frequencies (e.g., F₁, F₂, F₃). These individual peaks (e.g., P₁, P₂, P₃) corresponding to the various frequencies, such as the heart rate frequency F₁, the first harmonic of the heart rate frequency F₂, and the second harmonic of the heart rate frequency F₃ may then be used in an equation to calculate a PIVA Score. PIVA Score, representative of a patient's fluid status, is also a corollary for pulmonary capillary wedge pressure. Because pulmonary capillary wedge pressure is an indicator of fluid status (e.g., hypervolemia or hypovolemia), the PIVA Score, likewise, is representative of a patient's fluid status.

In an embodiment, the equation to calculate PIVA Score is represented by the following:

$$PIVA Score = {c}_{3} tanh ( \frac{{g}_{0} + {g}_{1} {mag}^{f 1} + {g}_{2} {mag}^{f 2} + {g}_{3} {mag}^{f 3}}{2} ) + {c}_{2} tanh ( \frac{{h}_{0} + {h}_{1} {mag}^{f 1} + {h}_{2} {mag}^{f 2} + {h}_{3} {mag}^{f 3}}{2} ) + {c}_{1} tanh ( \frac{{i}_{0} + {i}_{1} {mag}^{f 1} + {i}_{2} {mag}^{f 2} + {i}_{3} {mag}^{f 3}}{2} ) + {c}_{0}$$

Each of c₀, c₁, c₂, c₃, g₀, g₁, g₂, g₃, h₀, h₁, h₂, h₃, i₀, i₁, i₂, and i₃ are constants. Each of mag^{f1}, mag^{f2}, and mag^{f3} represents the individual magnitudes of each of the respective frequencies (e.g., F₁, F₂, F₃). These magnitudes are also commonly referred to herein as peaks of frequencies. For example, mag^{f1} may also be referred to as peak P₁ herein, associated with heart rate frequency F₁. Similarly, for example, mag^{f2} may also be referred to as peak P₂ herein, associated with first harmonic frequency F₂. Similarly, for example, mag^{f3} may also be referred to as peak P₃ herein, associated with second harmonic frequency F₃. For example, and with reference to FIG. 4B, P₁, referred to in the PIVA Equation as mag^{f1}, is the magnitude of the heart rate frequency (F₁), P₂, referred to in the PIVA Equation as mag^{f2}, is the magnitude of a first harmonic frequency (F₂), and P₃, referred to in the PIVA Equation as mag^{f3}, is the magnitude of a first harmonic frequency (F₃).

Evaluation unit 118 calculates the PIVA Score, which is unitless. In a related embodiment, PIVA system 100 displays the PIVA Score (e.g., via monitor 120). By calculating PIVA Score, a patient's fluid status may be readily determined (e.g., hypovolemia, hypervolemia, or euvolemia). Preferably, the calculated PIVA Score has an agreement with pulmonary capillary wedge pressure of ±8 mmHg with limits of agreement of 95% confidence interval.

In an embodiment, additional peak magnitudes corresponding to various frequencies (e.g., P₄ corresponding to F₄, a third harmonic frequency) may also be used in calculating the PIVA Score (e.g., implementing additional constants as well) for greater accuracy in calculation.

In an embodiment, the calculation or measurement can be directly related to a magnitude or change in magnitude of a harmonic peak (P_{H}) associated with a harmonic frequency (F_{H}), such as a change from a magnitude at a prior time when the patient's hemodynamic state was known (e.g., a baseline measurement prior to surgery). As yet another example, heart rate variability may be determined based upon changes in the heart rate frequency (F₁) over time or by measuring the width of a portion of the frequency-domain representation of the PVP signal associated with the heart rate peak (P₁) (e.g., the full width at half maximum).

In some embodiments, the patient status metric may be determined based upon a comparison of magnitudes associated with different frequency peaks (F_{N} and F_{M}) based upon the same plurality of data values (i.e., for the same evaluation window). For example, a ratio of the magnitudes associated with heart rate and first harmonic frequencies F₁ and F₂ may be used to determine a hemodynamic metric for the patient, such as a systemic vascular resistance or a blood volume score. Such ratios may be particularly useful in normalizing magnitudes associated with harmonic frequencies (F_{H}) to obtain more robust and more accurate patient status metrics. Similarly, ratios between magnitudes of the frequency-domain representation of the PVP signal associated with different harmonic frequencies (e.g., F₂ and F₃) may be used to determine hemodynamic state of the patient 102 (e.g., blood volume). In further embodiments, the patient status metric may be determined based upon a comparison of magnitudes associated with the same one or more frequencies (F_{N}) of peaks (P_{N}) determined for different pluralities of data values (i.e., for different evaluation windows). For example, analysis of a change in the absolute or relative magnitude associated with heart rate frequency F₁ over time may be used to determine a hemodynamic metric. Information regarding the patient status may be stored in a memory, presented to a user via the monitor 120, or used by the response unit 116 to generate and implement a response (e.g., presenting an alarm or controlling the operation of the fluid source 110), including any of the responses discussed further below.

In further embodiments, additional information regarding the patient may be used in determining some patient status metrics, or such additional information may be monitored for use with the patient status metrics. For example, information regarding a patient position or movement (e.g., a patient movement metric) may be separately monitored to provide context for the patient status metric or to supplement the patient status metric. To this end, additional patient metrics may be separately monitored by additional sensors 150 collecting data regarding positions or movements of the patient 102, or multiple patient metrics may be determined by analysis of the PVP signal monitored via the pressure sensor 112. For example, a sudden shift in a patient metric derived from PVP measured by the pressure sensor 112 (such as heart rate frequency F₁ or associated magnitude P₁) and a spike in measured acceleration from an addition sensor 150 may be combined to determine the patient has likely fallen. As another example, a frequency-domain analysis of the PVP signal may and a time-domain analysis of the PVP signal (e.g., waveform analysis or pattern detection) may both be performed to generate patient metrics, which may then be combined or analyzed together to evaluate the patient status. The additional patient metrics may be evaluated to verify appropriateness of responses to changes in a patient status metric. Thus, if a patient status metric indicates a possibility of a transient condition at the same time as an additional patient metric indicates a patient movement, the patient status metric may be determined to be the result of the patient movement, so no response may be required. Alternatively, if the additional patient metric confirms a patient status metric that indicates a patient movement or exertion for a patient who requires ambulatory assistance, an alert may be generated to warn responsible personnel that that patient may be attempting to walk without assistance. In some embodiments, the additional information may include information indicating a patient condition or limitations, such as patient condition information entered by a physician or nurse.

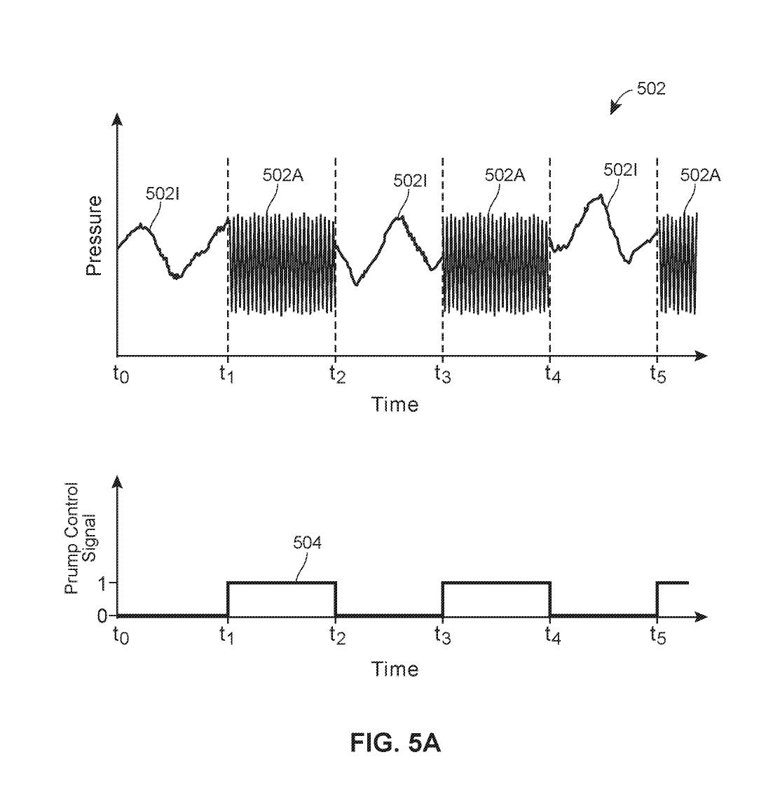

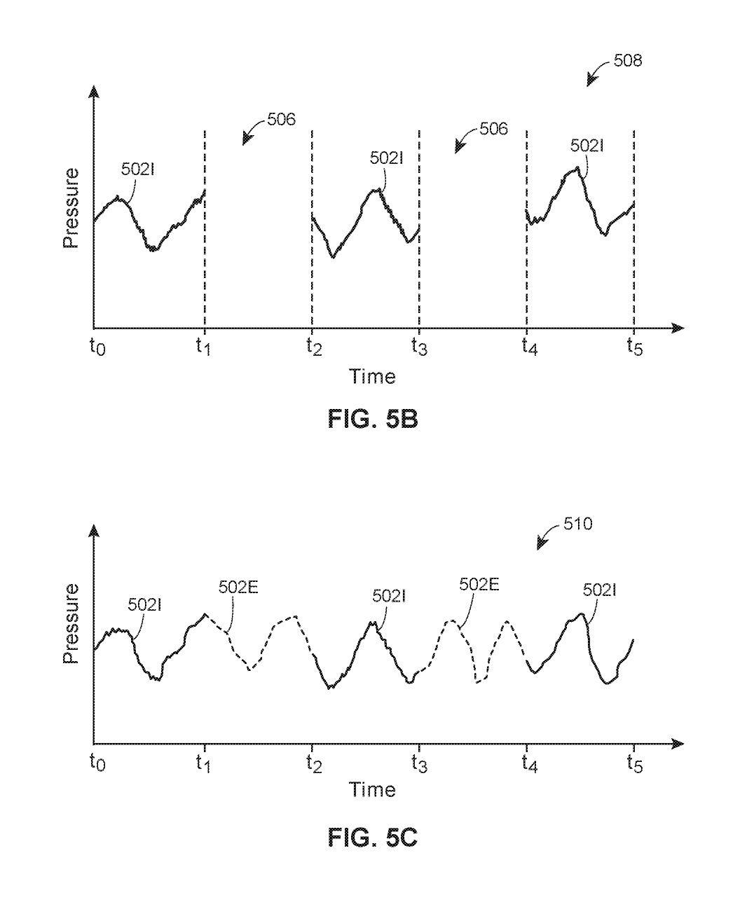

FIGS. 5A-C illustrate exemplary charts of time-domain representations of a PVP signal that includes noise artifacts, such as from operation of a pump 111 or other fluid source 110. The exemplary charts illustrate various stages or types of processing that may be performed by the analysis component 114. FIG. 5A illustrates a PVP data signal 502 that includes both inactive segments 502I associated with inactive time segments during which the pump 111 is not operating and active segments 502A associated with active time segments during which the pump 111 is operating. To show the effect of pump activity on the PVP data signal 502, FIG. 5A further illustrates operation of the pump 111 by charting the pump control signal 504 on the same time scale. The pump control signal 504 is illustrated for simplicity as a binary signal, with a value of “1” indicating active pumping and a signal of “0” indicating inactivity. In alternative embodiments, however, alternative types of pump control signals may be used to control the power or mode of operation of the pump 111.

As illustrated in FIG. 5A, the pump 111 is not operating during the first inactive time segment between times t₀ and t₁, so the values of the PVP signal 502 during this time segment form an inactive-pump PVP signal 502I. The inactive-pump PVP signal 502I represents PVP measurements corresponding to pressure in the circulatory system of the patient 102 without interference from the pump 111. As such, the inactive-pump PVP signal 502I is similar to the time-domain PVP signal 402, described above. Thus, the values of the inactive-pump PVP signal 502I may be used to perform further analysis according to PIVA or other frequency-domain methods, as discussed herein. As further illustrated, the pump 111 is operating during the first active time segment between times t₁ and t₂, immediately following the first inactive time segment. The values of the PVP signal 502 during the first active time period form an active-pump PVP signal 502A, the values of the which include noise artifacts from the operation of the pump 111. The noise artifacts of such active time segments inhibit PIVA and other related analysis, so it is useful to remove, replace, or adjust the active-pump PVP signal 502A prior to further analysis. Additional second and third inactive time segments associated with inactive-pump PVP signals 502I during which the pump 111 is not operating are further illustrated between times t₂ and t₃ and between times t₄ and t₅. An additional active time segment associated with active-pump PVP signals 502A during which the pump 111 is operating is illustrated between times t₃ and t₄. Although the active time segments and inactive time segments are illustrated as adjacent in time in the exemplary chart, some embodiments may include transition periods that are neither part of any inactive time period nor part of any active time period.

FIG. 5B illustrates an exemplary cleaned PVP signal 508 comprising only the inactive-pump PVP signals 502I. The exemplary cleaned PVP signal 508 may be generated by simply removing data values associated with the active time segments, leaving gaps 506 in the cleaned PVP signal 508. In order to remove the active time segments, the analysis component 114 may first identify one or more of either or both of active time segments or inactive time segments. In some embodiments, information from the pump 111 (such as the pump control signal 504) may be used to identify active time segments or inactive time segments. In preferred embodiments, however, the analysis component 114 may identify the active time segments or inactive time segments based upon the values of the PVP signal 502. The analysis component 114 may identify the active time segments or inactive time segments based upon magnitudes of the values or changes in the values of the PVP signal 502, as discussed further below.

Once generated, the cleaned PVP signal 508 may be directly analyzed according to the methods described herein, or the PVP signal 508 may be further adjusted further prior to transformation to the frequency domain. For example, the cleaned PVP signal 508 may be adjusted to remove the gaps 506 by aligning the inactive-pump PVP signals 502I to be partially overlapping based upon a periodicity of the inactive-pump PVP signals 502I. As another example, the cleaned PVP signal 508 may be adjusted to fill the gaps 506 with estimated values based upon the inactive-pump PVP signals 502I, as illustrated in FIG. 5C. Alternatively, instead of estimating the gaps 506, the inactive-pump PVP signals 502I can be connected via other means, such as via straight lines connecting an end point of one inactive pump PVP signal 502I to a start point of a second inactive pump PVP signal 502I (e.g., a straight line across gap 506). Although the inactive-pump PVP signal 502I associated with a single inactive time segment may be sufficient for frequency-domain analysis of patient status metrics if the inactive time segment is of sufficiently long duration, the inactive time segments may be of too short durations to allow accurate analysis. In such instances, combining a plurality of inactive-pump PVP signals 502I over a corresponding plurality of inactive time segments facilitates further analysis by providing more data for evaluation. Even when individual inactive time segments are sufficiently long to allow frequency analysis, the accuracy may be improved by adding additional data values associated with additional inactive time segments.

FIG. 5C illustrates an exemplary adjusted PVP signal 510 comprising the inactive-pump PVP signals 502I and estimated PVP signals 502E to fill the gaps 506. The values of the estimated PVP signals 502E may be estimated based upon the values of the inactive-pump PVP signals 502I of the cleaned PVP signal 508, as discussed further below. By filling the gaps 506 with the estimated PVP signals 502E, the resulting adjusted PVP signal 510 may be better suited for some types of further analysis. Specifically, the adjusted PVP signal 510 represents a comprehensive time series of data without noise artifacts from operation of the pump 111, which may be analyzed without further adjustment for the effects of pumping. It should be noted that the adjusted PVP signal 510 may be obtained from the measured PVP signal 502 alone, without reference to extrinsic data regarding the pump 111. Thus, extrinsic data regarding times of pump operation (e.g., time periods of pump operation) or characteristics of pump operation (e.g., pump speed, pump volume, or models of noise artifacts generated by the pump) are not needed in order to generate the adjusted PVP signal 510.

Although FIG. 5C illustrates the estimated PVP signals 502E as only filling the gaps 506 created by removing the active-pump PVP signals 502A, some embodiments may include estimating the entire adjusted PVP signal 510. In such embodiments, both the active-pump PVP signals 502A and the inactive-pump PVP signals 502I may be replaced with estimated PVP signals 502E to generate the adjusted PVP signal 510. Although such approach may reduce accuracy of the analysis in some respects by replacing measured values of the inactive-pump PVP signals 502I with estimated values of the estimated PVP signals 502E, the approach may better facilitate further analysis by eliminating discontinuities at the boundaries between the active and inactive time segments (i.e., at times t₁, t₂, t₃, and t₄). In yet further embodiments, discontinuities may be addressed by adjusting values of one or more of the inactive-pump PVP signals 502I or the estimated PVP signals 502E that occur near the boundaries between the active and inactive time segments to smooth the transitions. In any case, the active-pump PVP signals 502A are excluded from the adjusted PVP signal 510 and are replaced with the estimated PVP signals 502E.

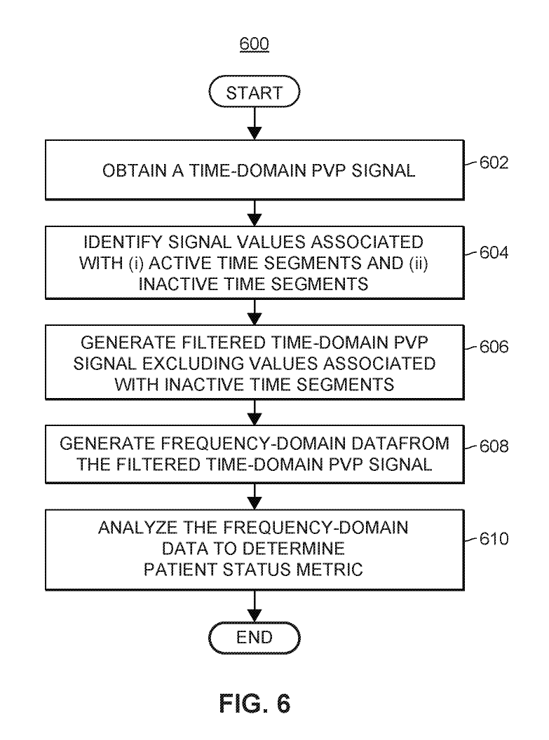

FIG. 6 illustrates a flow diagram of an exemplary pressure signal filtering method 600 for removing noise artifacts related to operation of a medical device from a signal corresponding to the PVP of the patient 102. The filtering method 600 may be implemented by the evaluation unit 118 to obtain, filter, and analyze a PVP signal to determine a patient status metric. Noise artifacts from the operation of a pump 111, other fluid source 110, or similar medical device can obscure ordinary PVP measurements during operation. For analytical methods such as PIVA, these noise artifacts must be removed or otherwise addressed prior to further processing in order to obtain accurate metrics. In contrast with other methods of addressing device noise artifacts, the filtering method 600 identifies and removes signal values associated with active time segments from a PVP signal containing active time segments of device operation and inactive time periods when the device is inactive. To do this, a time-domain PVP signal (such as PVP signal 502) is obtained and processed to remove signal values associated with active time segments (such as active-pump PVP signals 502A) to generate a filtered time-domain PVP signal (such as cleaned PVP signal 508 or adjusted PVP signal 510). The filtered time-domain PVP signal may then be transformed to the frequency domain and analyzed according to the methods discussed herein to determine one or more patient status metrics.

The filtering method 600 begins by obtaining a time-domain PVP signal from measurements associated with pressure in a peripheral vein of the patient 102 (block 602). The time-domain PVP signal may be directly generated by the pressure sensor 112 or may be derived from sensor measurements, as discussed elsewhere herein. As also described elsewhere herein, the time-domain PVP signal may be obtained by monitoring the pressure sensor 112 or by accessing a stored PVP data signal. In some embodiments, the evaluation unit 118 may monitor and record data from a transducer to generate the time-domain PVP signal. The time-domain PVP signal may include one or more of each of the following: (i) active time segments during which the pump 111 is operating (i.e., actively pumping) and (ii) inactive time segments during which the pump 111 is not operating (i.e., not actively pumping). The active time segments and the inactive time segments may alternate periodically or aperiodically. Although the pump 111 may be configured to operate in such a manner as to inherently produce both active and inactive time segments during the ordinary course of use, the active time segments are periods during which the pump 111 is generating noise artifacts by active operation, while the inactive time segments are periods during which the pump 111 is not generating significant noise artifacts by passive or inactive operation (e.g., a rest period between cyclical pumping). To enable further analysis of the measured PVP, the evaluation unit 118 may identify and filter the active and inactive time segments.

The filtering method 600 may, therefore, identify values of the time-domain PVP signal associated with the active time segments or the inactive time segments (block 604). The evaluation unit 118 may automatically identify the active time segments, the inactive time segments, or both the active and inactive time segments based upon the values of the time-domain PVP signal. In preferred embodiments, the evaluation unit 111 may identify the time segments based solely upon analysis of the time-domain PVP signal, without reference to additional extrinsic information regarding the characteristics or operating status of the pump 111 that is not contained in or derived from the time-domain PVP signal (e.g., previously determined pump operating parameters or a control signal controlling operation of the pump). Thus, the evaluation unit 118 may identify the time segments in the same manner, regardless of the characteristics, configuration, or settings of the pump 111, and without requiring adjustments to or further configuration of the evaluation unit 118. In various embodiments, the evaluation unit 118 may automatically identify the time segments based upon magnitudes of the values of the time-domain PVP signal or based upon changes in magnitudes of the values of the time-domain PVP signal. The values may be analyzed individually or in sets containing a plurality of values, according to one or more set metrics applied to the sets.

For individual values of the time-domain PVP signal, each of a plurality of values may be compared to one or more threshold levels to determine whether the value is associated with a time within an active time segment or an inactive time segment. For example, values above an upper threshold level may be identified as being associated with an active time segment, or values below a lower threshold level may be identified as being associated with an inactive time segment. The values may be grouped based upon such comparisons to identify the active and inactive time segments. While the upper and lower threshold levels may be identical in some embodiments, they may be distinct levels in other embodiments. When distinct, an indeterminate range exists in which values cannot be assigned to either active time segments or inactive time segments. Such indeterminate values may be further analyzed based upon the surrounding time segments to determine whether such indeterminate values belong to active time segments, inactive time segments, or transition time segments. In some embodiments, outlier values may be discarded or identified as being part of an active or inactive time segment based upon values surrounding such outlier values in time (i.e., preceding and following the outlier value). In order to remove the noise artifacts more completely, transition time segments may be treated as active time segments in some embodiment for the purpose of generating the filtered time-domain PVP signal.

For sets of values of the time-domain PVP signal, each set may be analyzed using one or more set-metrics to determine whether the set is associated with an active or inactive time segment. In preferred embodiments, each set contains values of the time-domain PVP signal that are adjacent in time, forming a time series of values of the PVP signal. Thus, each set is associated with a set-specific time period and comprises values associated with times within the set-specific time period. The set-specific time periods of the sets may cover fixed durations or may be of variable durations, and the set-specific time periods may be overlapping or non-overlapping. The sets may include sampled values from the time-domain PVP signal, or the sets may include all values of the time-domain PVP signal associated with times within the set-specific time periods of the corresponding sets. In particularly preferred embodiments, the set-specific time periods can be non-overlapping but adjacent sets covering all time periods within an analysis time period for which data is available for an uninterrupted duration of the time-domain PVP signal, such that each value of the time-domain PVP signal during the analysis time period is in exactly one of the sets. Thus, the active or inactive time segments may be identified as collections of one or more sets by identifying the sets as being associated with either active or inactive time segments.

To identify a set as being associated with an active or inactive time segment, the values of the time-domain PVP signal within the set may be evaluated using one or more set-metrics. The set-metrics may include functions that determine an average value, a maximum value, a minimum value, a distance between maximum and minimum values, an average change between values (or absolute value thereof), a variance of the set, or another metric of the values in the set. Once a set-metric has been determined by evaluating the values of a set, the set-metric may be compared against a set-threshold level associated with the set-metric to identify the set as being associated with an active time segment or an inactive time segment. For example, sets may be identified as being associated with active time segments when a set-metric is above the set-threshold level for the set-metric or being associated with inactive time segments when a set-metric is below the set-threshold level for the set-metric.

In some embodiments, the set-metric may determine changes between values within a set, such as a rate of change. Such rate of change may be an average rate of change, a maximum rate of change, or other measures of changes between the values. Set-metrics regarding changes or rates of change between values may be used to determine beginning times or ending times of active or inactive time segments by comparison against a threshold associated with the starting or stopping of active pumping. PVP may spike when the pump 111 begins active pumping at the start of an active time segment and drop precipitously when the pump 111 stops active pumping at the end of the active time segment. Thus, large and rapid changes in the values of the time-domain PVP signal may be used to identify beginnings or ends of the active and inactive time segments. For example, the beginning time of an active time segment may be identified by determining a change or rate of change set-metric is above a pumping start threshold, and the beginning time of an inactive time segment may be identified by determining a change or rate of change set-metric is below a pumping stop threshold. Active and inactive time segments may then be identified based upon such beginning or ending times.

Once the active and inactive time segments are identified in the time-domain PVP signal, the evaluation unit 118 may generate a filtered time-domain PVP signal (block 606). The filtered time-domain PVP signal may be an adjusted PVP signal 510 having estimated PVP signals 502E (as illustrated in FIG. 5C) or may instead be a cleaned PVP signal 508 that simply removes the active-pump PVP signals 502A (as illustrated in FIG. 5B). The filtered time-domain PVP signal is generated based upon the time-domain PVP signal and excludes the values of the time-domain PVP signal associated with the active time segments. In contrast to other methods that attempt to correct for pump noise artifacts by estimating and removing the noise artifacts themselves, the filtering method 600 estimates what the PVP signal would have been if the pump 111 had not been operating.

As illustrated above in the cleaned time-domain PVP signal 508, the filtered time-domain PVP signal may be generated by removing the values associated with one or more identified active time periods from the time-domain PVP signal. Where the time-domain PVP signal comprises a sequential time series of discrete values, the filtered time-domain PVP signal may be generated by removing those values identified by their corresponding times as falling within an active time segment, thereby leaving one or more sequential time series of discrete values corresponding to times falling within inactive time segments. In some embodiments, the filtered time-domain PVP signal may further be adjusted or normalized before further analysis. For example, the remaining values associated with inactive time segments may be stitched together to avoid having gaps in the filtered time-domain PVP signal (such as the gaps 506). To do this, corresponding values within each of a plurality of inactive time segments may be identified, and the inactive time segments may be combined by aligning these identified corresponding values. Thus, the beginning of one inactive time segment may be aligned with the end of the preceding inactive time segment such that the cycles (i.e., the cardiac cycles of the patient) are aligned. This may further require removing or blending overlapping values of one or both of the inactive time segments to produce an uninterrupted filtered time-domain PVP signal.

As illustrated above in the adjusted PVP signal 510, the filtered time-domain PVP signal may alternatively be generated by replacing the values associated with the one or more identified active time periods with substitute values. The substitute values are determined based upon the values associated with one or more inactive time segments in the time-domain PVP signal. Thus, the filtered time-domain PVP signal may be generated by combining the values of the time-domain PVP signal associated with the inactive time segments with the substitute values for the active time segments to produce an uninterrupted signal or time sequence of values. In some embodiments, the substitute values may be generated by estimating values for the active time segments based upon a model determined by regression analysis, principal component analysis, or similar techniques. The model parameters may be estimated by ordinary least squares regression on the values associated with the inactive time segments. In preferred embodiments, however, the model may be estimated by least cubes regression on the values associated with the inactive time segments, which produces improved results for PVP signals under many circumstances. In some embodiments, the substitute values may be adjusted near the boundaries between the active and inactive time segments in order to smooth the transitions between the inactive time segment values and the substitute values. In further embodiments, the substitute values may be estimated for both the active and inactive time segments, in which case the values of both may be replaced by the estimated substitute values to generate the filtered time-domain PVP signal. Such filtered time-domain PVP signals may be beneficial in some instances, inasmuch as such signals avoid breaks or discontinuities of the signal at boundaries between the active and inactive time segments.

Once the filtered time-domain PVP signal has been generated for one or more time periods (such as the evaluation windows discussed above), the evaluation unit 118 may further analyze the data by generating frequency-domain PVP data from the one or more filtered time-domain PVP signals (block 608). In a manner similar to that discussed elsewhere herein, a time-frequency transform (such as FFT) may be applied to the filtered time-domain PVP signals to generate frequency-domain PVP data as a representation of PVP in the frequency domain after filtering to remove the noise artifacts from operation of the pump 111. Such frequency-domain PVP data may be generated as frequency distributions associated with the one or more filtered time-domain PVP signals. By using the filtered time-domain PVP signal to generate the frequency-domain PVP data, PVP can be analyzed for patients connected to cyclically operating pumps 111, despite the noise artifacts generated by pump operation. If the pump 111 is directly connected to the patient's circulatory system, the methods described herein enable analysis at rates of operation up to the point at which the inactive time segments become too short and too infrequent for reliable filtering (e.g., approximately 250 cc/minute for most adult patients with typical heart rate and respiratory rate using an infusion pump such as the SIGMA Spectrum® infusion system produced by Baxter International Inc.). In an embodiment, the evaluation unit 118 further normalizes the frequency-domain PVP data. For example, the evaluation unit 118 may normalize frequency-domain PVP data to take into account inactive time segments. The frequency-domain data may then be further analyzed to determine one or more patient status metrics (block 610). Such frequency-domain analysis may include analysis of the frequencies or magnitudes of frequency peaks (F_{N}), as discussed in further detail elsewhere herein. In some embodiments, this may include comparing frequency-domain PVP data to determine changes in patient status metrics.

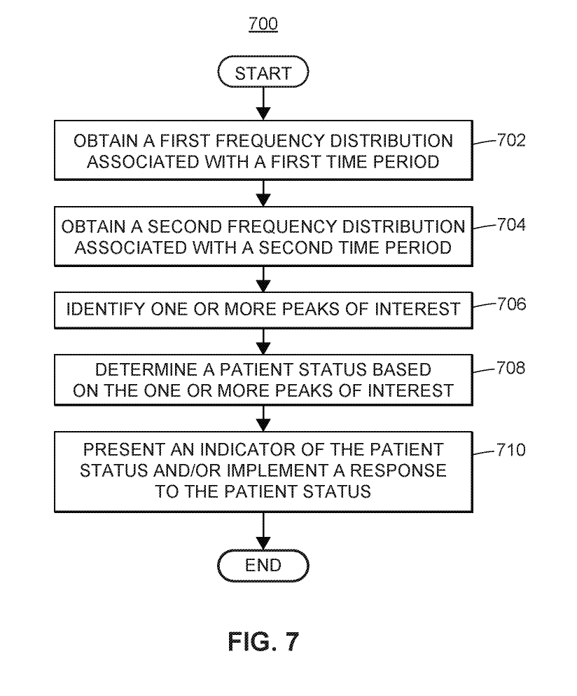

Because comparisons of changes in frequencies and associated magnitudes are particularly useful for monitoring patient condition via patient status metrics, a discussion of such comparisons is next described. Similar methods of comparison of metrics of the PVP signal in the time-domain across multiple time periods may likewise be performed to monitor patient condition in further embodiments. FIG. 7 illustrates an exemplary PIVA comparison method 700 for identifying changes in a patient status based upon comparison of frequency-domain representations of PVP signals associated with different times. The PIVA comparison method 700 may be implemented by the evaluation unit 118 and the response unit 116 to determine and respond to changes in patient status between time periods. For example, the evaluation unit 118 may determine and compare frequency-domain representations of PVP based upon electronic pressure signals received during multiple time periods to determine changes in patient metrics, such as blood pressure, blood volume, respiration, position or movement, or systemic vascular resistance. Specifically, the evaluation unit 118 may compare relative or absolute magnitudes associated with frequencies (F_{N}) of peaks (P_{N}) in the frequency distributions determined for each time period to identify changes in patient status that may be used by the response unit 118 to determine and implement response actions.

The exemplary method 700 begins by obtaining a first frequency distribution associated with a first time period (block 702) and a second frequency distribution associated with a second time period (block 704). Each of the first and second frequency distributions may be generated as the frequency-domain data corresponding to the plurality of data values from the PVP data signal by the method 300 or the filtering method 600, as described above. The first and second time periods may correspond to first and second evaluation windows, as discussed above, each evaluation window being associated with a plurality of data values sampled or received by the evaluation unit 118. The data values for each of the first and second evaluation windows may be stored in a volatile or non-volatile memory until needed by the evaluation unit 116 to generate the frequency distributions, as discussed above. Alternatively, the frequency distributions or information associated therewith (e.g., frequency peaks and associated magnitudes) may be stored directly for comparison. In some embodiments, the first and second frequency distributions may be frequency-domain representations of the PVP signals from the sensor 112 for time periods of fixed duration beginning at times separated by a predetermined interval. For example, the method 700 may be implemented on a rolling basis (i.e., periodically or when new PVP data becomes available) during real-time monitoring of the patient 102 by comparing the magnitudes of frequency peaks (F_{N}) of frequency distributions generated for first and second evaluation windows during patient monitoring. The first and second periods may be partially overlapping, adjacent in time, or separated by an intervening period.

The evaluation unit 116 may next identify one or more peaks of interest for determining the patient status metrics (block 706). The peaks of interest may be identified in either or both of the first and second frequency distributions. In some instances, the one or more peaks of interested may be determined based upon peaks (P_{N}) in a baseline frequency distribution generated for the patient 102, which may be the first frequency distribution or an additional prior frequency distribution. The baseline frequency distribution may, for example, be determined prior to scheduled surgery to establish a baseline for later patient status monitoring. The peaks of interest may be identified based upon the associated frequencies (F_{N}), such as by identifying the respiratory frequency (F₀) or the heart rate frequency (F₁). In some embodiments, the peaks of interests may include a plurality of such peaks, such as the peaks (P₂) and (P₃) associated with the first harmonic frequency (F₂) and the second harmonic frequency (F₃). Under some conditions, not all peaks of interest may be identifiable in both frequency distributions. For example, during an acute failure of the circulatory system, systemic vascular resistance may markedly decrease and peaks associated with the harmonic frequencies (F₂, F₃, . . . F_{N}) may not be discernible. Thus, the peaks associated with the harmonic frequencies (F₂, F₃, . . . F_{N}) may be identifiable in the first frequency distribution but not in the second frequency distribution. Nonetheless, a change in magnitude of the frequency distributions at the harmonic frequencies (F₂, F₃, . . . F_{N}) may be determined by comparison of the first and second frequency distributions.

Based upon the identified one or more peaks of interest, the evaluation unit 116 may further determine a patient status (or a change in patient status) by a comparison of the first and second frequency distributions (block 708). Determining the patient status may include a comparison of the magnitudes associated with the same one or more frequencies (F_{N}) between the first and second frequency distributions, a comparison of the values of a function of a plurality of magnitudes associated with frequencies between the first and second frequency distributions (e.g., a comparison of ratios of peak magnitudes), a comparison of frequencies (F_{N}) associated with one or more peaks (P_{N}) between the first and second frequency distributions (e.g., a change in the respiratory frequency or heart rate frequency), or a comparison of other metrics associated with patient status. In some embodiments, the patient status may be determined based upon a change in a metric beyond a threshold level. For example, a decrease in the magnitude associated with the heart rate frequency (F₁) in the second frequency distribution below 80% of the corresponding magnitude associated with the heart rate frequency (F₁) in the first frequency distribution may indicate hypovolemia in the patient 102. As another example, a decrease in the ratio of the magnitude associated with the first harmonic frequency (F₂) to the magnitude associated with the heart rate frequency (F₁) between the first and second frequency distributions beyond a predetermined threshold may indicate hypervolemia or hypovolemia, depending upon whether and how the magnitude associated with the heart rate frequency (F₁) changes. Comparisons of particular interest are discussed in further detail elsewhere herein.

A comparison involving one or more of the harmonic frequencies (F_{H}) is of particular interest regarding patient hemodynamic state or blood volume. Because the frequency distribution values associated with harmonic frequencies (F_{H}) are more sensitive to changes in blood volume than the values associated with the heart rate frequency (F₁), monitoring changes in the values associated with harmonic frequencies (F_{H}) may provide an earlier or clearer indication of patient hemodynamic state. For example, a sharp increase or decrease in the magnitude of the value of the frequency distribution associated with the first harmonic frequency (F₂) (or other harmonic frequency) may be more pronounced than the corresponding change in the values associated with the heart rate frequency (F₁) in the same patient at the same time. Thus, blood volume metrics may be generated using the harmonic frequencies (F_{H}). Such metrics may be determined as functions of the harmonic frequencies (F_{H}), ratios of the frequency values of the harmonic frequencies (F_{H}), magnitudes associated with the harmonic frequencies (F_{H}), ratios of the magnitudes associated with the harmonic frequencies (F_{H}), or changes in any of these. Such changes may be measured against a baseline or against a previously determined value at a fixed interval in time prior to the current values. In some embodiments, the frequencies or magnitudes associated with the harmonic frequencies (F_{H}) may be compared against other relevant values, such as frequencies or magnitudes associated with the respiratory rate frequency (F₀) or the heart rate frequency (F₁). For example, one or more harmonic frequencies (F_{H}) may be normalized by comparison against the heart rate frequency (F₁). Such normalized value may be determined as a ratio of the magnitudes and may be used as a blood volume metric to evaluate the hemodynamic state of the patient 102. Other similar blood volume metrics based at least in part upon the frequency and magnitude values of the one or more harmonic frequencies (F_{H}) may be determined and used to evaluate the hemodynamic state of the patient 102 in various embodiments.

Once patient status has been determined, the response unit 116 may determine whether a response is required and cause any required response to be implemented (block 510). This may include determining a patient condition based upon the patient status metric. Additionally, or alternatively, the evaluation unit 118 or the response unit 116 may cause an indicator of the determined patient status to be stored or presented via the monitor 120 (block 510). If the response unit 116 determines a response is required, the response unit 116 may further determine one or more responses that are appropriate to address the identified patient status. Such responses may include generating an alarm or other warning that the patient status is abnormal, which may include information regarding the patient condition. An alarm or warning may be presented via the monitor 120 or may be communicated to another device for presentation. The alarm or warning may include a recommendation of one or more actions to take in response to the patient status. For example, the recommendation may include an adjustment to a fluid therapy for the patient 102, which may include a recommendation to administer one or more vasopressors or vasodilators. Such recommendation may be determined by the response unit 116 as part of the required response. In some embodiments, this may include sending an electronic communication to a user device (e.g., a workstation or mobile device used by a physician, nurse, or technician to monitor patient condition).

The responses may similarly include controlling the fluid source 110 to adjust fluid flow to the patient 102. The fluid source 110 may be controlled to increase or reduce the rate of fluid flow to the patient 102, including starting or stopping fluid flow. In some embodiments, the response may include controlling the fluid source 110 (or a device connected thereto) to administer one or more drugs to the patient 102. For example, the fluid source 110 may be controlled to administer one or more vasopressors or vasodilators in a fluid delivered to the peripheral vein 108 via the IV tube 104 and venous access device 106. Where the fluid source 110 includes a pump, the response may include controlling the operation of the pump, such as by increasing or decreasing pump speed, flow rate, or mode of operation, as well as starting or stopping the pump. In some embodiments, the fluid source 110 may be controlled to administer a quantity of a drug to the patient 102 via the fluid. For example, the fluid source 110 may be controlled to add a quantity of the drug to the fluid. Additional embodiments of specific analysis and response methods utilizing the PIVA system 100 are further described elsewhere herein in greater detail.

### The PIVA Module

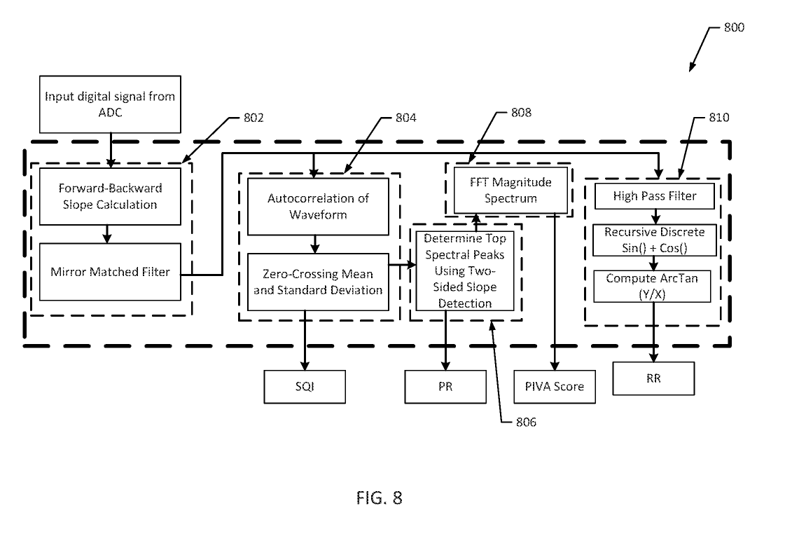

The PIVA system 100 may perform several signal filtering and signal processing steps (e.g., to remove the noise artifacts from a physiological signal, to perform FFT on a physiological signal, to calculate the PIVA Score, via the equation previously disclosed herein, as a corollary to pulmonary capillary wedge pressure, and other related functions). In an embodiment, the PIVA system 100 performs these steps, and others, via the PIVA module 800. Although the PIVA module 800 is described with reference to the block diagram illustrated in FIG. 8, it will be appreciated that many other configurations and methods of performing the acts associated with PIVA module 800 may be used. For example, the order of some of the blocks may be changed, certain blocks may be combined with other blocks, and some of the blocks described may be optional.

As illustrated in FIG. 8, the PIVA module 800 includes a noise module 802, a signal quality index module 804, a pulse rate module 806, an FFT module 808, and a respiratory rate module 810.

The PIVA module 800 receives at least one input. For example, the PIVA module 800 may receive a digital signal from an analog-digital converter. The digital signal may be representative of a patient physiological parameter, such as a patient's peripheral intravenous pressure. It should be appreciated that many other physiological parameters are contemplated, such as other invasive venous pressures, invasive arterial pressures, noninvasive venous pressure, noninvasive arterial pressures, and other similar parameters. In an example, the digital signal is derived from a medical device, such as a pressure transducer that is in fluid communication with the patient's vein.

Likewise, the PIVA module 800 delivers outputs. For example, the PIVA module 800 may output a signal quality index (SQI) related to the PIVA system 100, a respiratory rate (RR) of the patient, a pulse rate (PR) of the patient, and a PIVA Score of the patient.

### Noise Module

Responsive to receiving the digital signal, the PIVA module 800 may perform filtering and processing. In an embodiment, the digital signal is processed via noise module 802 to eliminate noise artifacts, such as those associated with the operation of a pump. For example, the noise module 802 may perform forward-backward slope calculations to identify segments of the digital signal where there is noise. In an embodiment, noise module 802 performs several processing steps to eliminate noise artifacts from a signal. In an embodiment, processing includes cascaded stack processing. This may advantageously provide for real-time processing and efficient decimation of recurrent feature calculations, block processes, filtering, and the like.

More specifically, the noise module 802 may evaluate the digital signal, identify a point where the positive slope of the signal is greater than a particular threshold (e.g., a signal spike), and characterize this portion of the digital signal as a noise start point. This may generally be characterized as slope based burst detection. Similarly, the noise module 802 may evaluate the digital signal, identify a point where the negative slope of the digital signal is less than a particular threshold (e.g., a signal drop) and characterize this portion of the signal as a noise end point. Slopes may be calculated by taking the derivative of the digital signal.

In an example, noise module 802 implements a sliding window stack size that is sufficient for local parameter estimation (e.g., for real-time processing). Noise module 802 determines the slope window size on each side of a peak within a particular stack (e.g., peaks typically associated with signal noise). For example, to calculate the slopes:

ForwardSlope=S{X[p-wdex]−X[p]}/(p-wdex)

BackwardSlope=S{X[p]−X[p-wdex]}(p-wdex)

Preferably, spacing between slope windows is tested for a wide range of pump rates. Noise module 802 may also calculate a symmetry point between the forward and backward slopes. The symmetry point may infer the peak-noise location. In an embodiment, high slope and/or high amplitude noise is detected.

The slope based burst detection is an adaptive input signal conditioning process, which provides for real-time noise cancellation. For example, noise module 802 identifies a noise-start and a noise-stop time, removes the signal between the noise-start time and noise-stop time (e.g., concatenates the signal). In other words, once a noise segment is identified (e.g., the signal portion between the noise start point and the noise end point), the noise module 802 may delete the segment from the digital signal (e.g., to produce a concatenated or segmented signal).

Likewise, for example, noise module 802 may also perform mirror-matched filtering to fill in gaps of the concatenated signal. More specifically, the signal range between the sign-adjusted forward and backward slopes greater than a threshold (e.g., the signal noise region) is replaced by a mirror image of the symmetrically split adjacent regions. In one embodiment, mirror-matched filtering involves filling in each gap from the front (e.g., from the noise end point) and the back (e.g., from the noise start point). In a different embodiment, mirror-matched filtering involves filling in the gap using prior digital signal data, which is stored in a memory (e.g., buffer memory). For example, noise module 802 retrieves buffer stack memory and fills synthetic data from the forward and/or reverse direction of the signal. In an embodiment, buffer and window size are optimized for pump rates from 25 Hz to 250 Hz.

Processing performed by noise module 802, including slope based burst detection and subsequent mirror matching, advantageously eliminates noise artifacts from signals. For example, with pump rates up to 250 mL per hour, noise module 802 has at least 0.74 seconds between pumping intervals; this is necessary for patients with low pulse rate to acquire appropriate signals. Preferably, the end result is a cleaned signal that has eliminated noise artifacts. After the noise module 802, the PIVA module 800 may perform additional processing on the cleaned signal.

### Signal Quality Index Module

In an embodiment, the cleaned signal may be processed via signal quality index module 804 to obtain the SQI related to the PIVA system 100. For example, the signal quality index module 804 may include autocorrelation of the cleaned signal (e.g., the waveform), which may include determination of both zero-crossing mean, and standard deviation of zero crossings. Zero crossings analysis may advantageously be used to calculate SQI. Responsive to processing via the signal quality index module 804, the PIVA module 800 may output the SQI.

More specifically, determining signal quality includes analyzing the autocorrelation of the signal. Autocorrelation may include putting the raw digital signal on top of itself (e.g., the raw digital signal on top of the cleaned signal). When there is a statistical spread in zero crossings, approximately the same as the zero crossing rate, the signal may be an unusable signal. For example, when the standard deviation of the zero crossings is similar to the number of zero crossing events, the signal may be unusable. To calculate Signal Quality:

ZCSD=Autozerocross−zerocrossSD

Signal Quality=sqrt(abs(ZCSD)/(autozerocross+zerocrossSD))

This calculated signal quality value may be displayed as a signal quality percentage and delivered as SQI.

In an embodiment, if the signal quality is determined to be “low” quality, a monitor in communication with PIVA module 800 will display a specific graphical user interface. For example, the monitor may indicate “Poor Signal Quality.” Similarly, the monitor may include signal quality troubleshooting recommendations. For example, the monitor may suggest to (1) check patient status, (2) check IV catheter for displacement, air, and kinks, (3) check pump rate to ensure it is below 250 mL per hour, (4) check for patient movement, (5) identify that the device may not be compatible for use with more than one infusion pump, and (6) flush and confirm that the IV catheter draws back.

### Pulse Rate Module

In a related embodiment, the cleaned signal may be processed via pulse rate module 806 to obtain the PR of the patient. For example, the pulse rate module 806 may determine top spectral peaks of the cleaned signal using two-sided slope detection. In an example embodiment, two-sided slope detection is a form of band pass filtering (e.g., high pass and/or low pass filters) implemented in either hardware or software. Responsive to processing via the pulse rate module 806, the PIVA module 800 may output the PR.

More specifically, processing includes cascaded stack processing. This may advantageously provide for real-time processing and efficient decimation of recurrent feature calculations, block processes, filtering, and the like.

In an embodiment, pulse rate module 806 implements autocorrelation processing for periodicity determination to compute pulse rate (also referred to herein as heart rate or HR). For example, pulse rate module 806 uses an 8192 sample block size, which may also be the stack buffer size that is processed in reverse order for correct periodicity features. Pulse rate module 806 may implement selectable overlapping intervals. As an example, the default interval may be a one second interval with 500 samples. Pulse rate module 806 may compute autocorrelation for lags (e.g., 0 to 4000, related to periodicities up to 8 seconds). Pulse rate module 806 may compute peak-associated lags (e.g., 17 peak-associated lags), using forward and backward slope detection as previously described. Pulse rate module 806 may filter zero-crossing periods and standard deviations. Pulse rate module 806 may compute filtered mean-spacing between sub-harmonics. Pulse rate module 806 may compute an HR estimate.

In an embodiment, pulse rate module 806 implements spectral processing (FFT) to determine HR. For example, pulse rate module 806 uses a 8192 point block size, which may preferably include no window function. Pulse rate module 806 may determine spectral peaks by forward and backward slope technique. Pulse rate module 806 may use the zeroth harmonic as a partial HR estimate. The spectral magnitude associated peaks, which are independent of autocorrelation inferred repetition rates, are accordingly identified. Spectral magnitude peak identification may be used to calculate respiratory rate or pulse rate (e.g., via one discrete peak) as well as volume index or patient fluid status (e.g., via multiple peaks). Discussion of spectral magnitude peak identification is included in the FFT Module section below.

In a related embodiment, pulse rate module 806 implements FFT to refine HRs previously determined via autocorrelation. In this embodiment, the HR initially calculated via autocorrelation is a partial HR estimate.

In another embodiment, pulse rate module 806 further computes heart rate variability (HRV) and HRV variability. For example, because pulse rate module 806 is performing peak detection over sliding windows, pulse rate module 806 can determine how data changes or varies, and thus determine HRV and HRV variability.

### FFT Module

In a related embodiment, the cleaned signal may be processed via FFT module 808 to obtain the PIVA Score of the patient. For example, the FFT module 808 may perform spectral analysis on the cleaned signal to obtain magnitudes. These FFT magnitude spectra may be used to calculate a PIVA Score (as described in greater detail below). Responsive to processing via the FFT module 808, the PIVA module 800 may output the PIVA Score.

More specifically, FFT module 808 is used to identify spectral magnitude peaks, which are subsequently used to calculate volume index (e.g., multiple peaks). In an embodiment, processing includes cascaded stack processing. This may advantageously provide for real-time processing and efficient decimation of recurrent feature calculations, block processes, filtering, and the like.

FFT module 808 implements spectral processing to identify spectral magnitude peaks. In an embodiment, identification of the individual magnitude peaks includes: utilizing the max found change in forward backward slope that is assisted by the guidance of the autocorrelation pulse rate, the magnitude peaks of the Fourier transformation are found.

In an embodiment, the equation to calculate PIVA Score is represented by the following:

PIVA Score=c₃ tan h(g₀+g₁mag^{f1}+g₂mag^{f2}+g₃mag^{f3}/2)+c₂ tan h(h₀+h₁mag^{f1}+h₂mag^{f2}+h₃mag^{f3}/2)+c₁ tan h(i₀+i₁mag^{f1}+i₂mag^{f2}+i₃mag^{f3}/2)+c₀

Each of c₀, c₁, c₂, c₃, g₀, g₁, g₂, g₃, h₀, h₁, h₂, h₃, i₀, i₁, i₂, and i₃ are constants. Each of mag^{f1}, mag^{f2}, and mag^{f3} represents the individual magnitudes of each of the respective frequencies (e.g., F₁, F₂, F₃). These magnitudes are also commonly referred to herein as peaks of frequencies. For example, mag^{f1} may also be referred to as peak P₁ herein, associated with heart rate frequency F₁. Similarly, for example, mag^{f2} may also be referred to as peak P₂ herein, associated with first harmonic frequency F₂. Similarly, for example, mag^{f3} may also be referred to as peak P₃ herein, associated with second harmonic frequency F₃. For example, and with reference to FIG. 4B, P₁, referred to in the PIVA Equation as mag^{f1}, is the magnitude of the heart rate frequency (F₁), P₂, referred to in the PIVA Equation as mag^{f2}, is the magnitude of a first harmonic frequency (F₂), and P₃, referred to in the PIVA Equation as mag^{f3}, is the magnitude of a first harmonic frequency (F₃)

Additional ways to determine the relationship between PIVA Score and patients' pulmonary capillary wedge pressure include the fitting of data was evolutionary algorithms to optimize a low complexity and low error solution as well as neural network mapping of the data with a training and validation set using nodes of hyperbolic tangential functions to create non-linear relationships between values.

In a related embodiment, FFT module 808 performs an algorithmic approach to calculating volume index. For example, FFT module 808 performs an initial least squares approach to analyze the individual magnitudes (e.g., F₁, F₂, F₃, etc.) and subsequently calculates a best-fit for volume index. The best-fit for volume index can, alternatively, be characterized as a best-fit for pulmonary capillary wedge pressure. Responsive to generating the best-fit, the FFT module 808 may use the best-fit for subsequent iterations to calculate volume index. In this example, subsequent iterations may allow for additional calculations of the PIVA score.

### Respiratory Rate Module

In an embodiment, the cleaned signal may also be processed via respiratory rate module 810 to obtain the RR of the patient. For example, the respiratory rate module 810 may filter the cleaned signal through a high-pass filter. The respiratory rate module may further perform recursive discrete analysis (e.g., sin( )+cos ( ) and computation of related ArcTan (y/x) to determine RR. Responsive to processing via the respiratory rate module 810, the PIVA module 800 may output the RR.

Determining respiratory rate may include using a digital linear FM discriminator based on differential phase angle filtering. Prior to this determination, pulse rate is calculated, as described above. The pulse rate data is then replicated. The respiratory rate module 810 applies a digital high-pass filter to the signal. For example, the high-pass filter isolates the respiratory rate frequency range and permits fitting of data to extract the respiratory rate.

More particularly, the input signal is high-passed filtered, for maximum volatility detection. The respiratory rate module 810 performs recursive filtering of quadrature:

Cosine(2*PI*n*k) and

Sin(2*PI*n*k)

and then calculates the filtered ArcTan( ) of filtered quadrature terms. The respiratory rate module 810 computes the derivative of the filtered ArcTan( ) angle. In an embodiment, the respiratory rate module 810 further performs light filtering of the derivative of the filtered ArcTan( ) angle. The respiratory rate module 810 may then estimate dominant baseband frequency. Multiplying the estimation by 60 provides a respiration rate on a per minute basis.

In a different embodiment, the RR of the patient is determined directly via the FFT signal. For example, as previously stated and with reference to FIG. 4B, under ordinary conditions, the peak (P₀) with the lowest frequency (F₀) corresponds to the respiration rate of the patient 102. Likewise, the peak (P₁) with the next-lowest frequency (F₁) corresponds to the heart rate of the patient 102. Thus, the RR (and the HR) of the patient can be readily determined directly via the magnitudes of the respective peaks: P₀ and P₁.

### The PIVA System

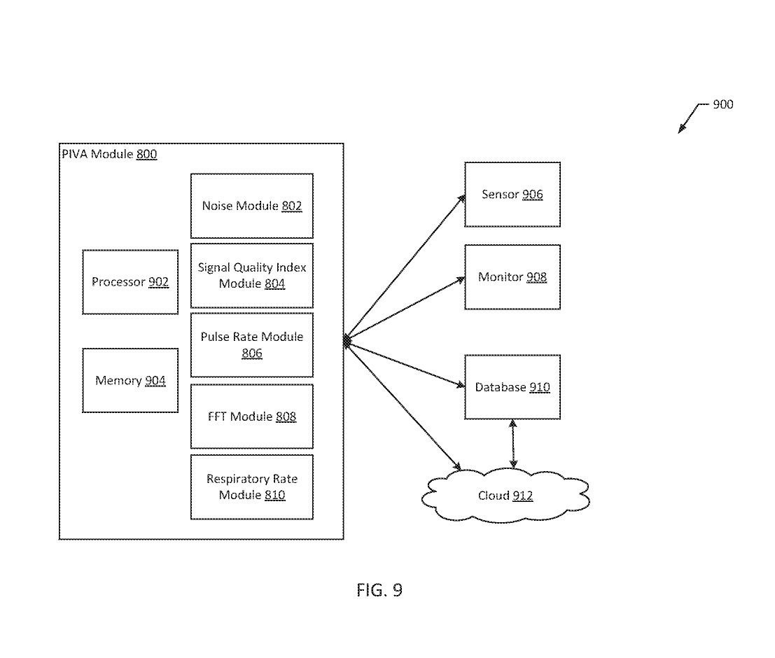

FIG. 9 illustrates a block diagram of an exemplary PIVA system 900, including the PIVA module 800 previously described herein. In addition to PIVA module 800, PIVA system 900 may include processor 902 and memory 904, running on PIVA module 800. For example, PIVA module 800 may include one or more physical processors 902 communicatively coupled to one or more memory devices 904.

Physical processor, such as processor 902, refers to a device capable of executing instructions encoding arithmetic, logical, and/or I/O operations. In one illustrative example, a processor may follow Von Neumann architectural model and may include an arithmetic logic unit (ALU), a control unit, and a plurality of registers. In an example, a processor may be a single core processor, which is typically capable of executing one instruction at a time (or process a single pipeline of instructions), or a multi-core processor, which may simultaneously execute multiple instructions. In another example, a processor may be implemented as a single integrated circuit, two or more integrated circuits, or may be a component of a multi-chip module (e.g., in which individual microprocessor dies are included in a single integrated circuit package and hence share a single socket). A processor may also be referred to as a central processing unit (CPU). Memory device, such as memory device 904, refers to a volatile or non-volatile memory device, such as RAM, ROM, EEPROM, or any other device capable of storing data. Local connections, including the connections between processor 902 and memory device 904, may be provided by one or more local buses of suitable architecture, for example, peripheral component interconnect (PCI).

Likewise, PIVA system 900 may include sensor 906 and monitor 908. For example, PIVA module 800 may be in communication with each of sensor 906 and monitor 908. Communication may be wired and/or wireless (e.g., WiFi, Bluetooth, and other related wireless protocols). In an example, sensor 906 is the pressure sensor 112 described in greater detail above. In an example, monitor 908 is the monitor 120 described in greater detail above. In an embodiment, PIVA module 800 is physically located within monitor 908.

Likewise, PIVA system 900 may include database 910 and cloud 912. For example, PIVA module 800 may be in communication with each of database 910 and cloud 912. Communication may be wired and/or wireless (e.g., WiFi, Bluetooth, and other related wireless protocols). In an example, database 910 includes electronic medical records stored on a hospital network. In an example, cloud 912 includes a remote storage location, which may be used to store physiological data and/or device information (e.g., PIVA module 800 performance statistics, software updates, and other related information).

In an embodiment, the PIVA system 900 displays an updated volume index via monitor 908 every 60 seconds. Preferably, the PIVA Score used to compute the volume index has an agreement with pulmonary capillary wedge pressure of ±8 mmHg with limits of agreement of 95% confidence interval.

In an embodiment, the PIVA system 900 displays an updated pulse rate via monitor 908 every 10 seconds. Preferably, the pulse rate has an agreement with the heart rate of ±10 beats per minute with limits of agreement of 95% confidence interval.

In an embodiment, the PIVA system 900 displays an updated respiration rate via monitor 908 every 10 seconds. Preferably, the respiration rate has an agreement with the respiratory rate of ±5 breaths per minute with limits of agreement of 95% confidence interval.

In an embodiment, the PIVA system 900 operates in conjunction with an external medical device. For example, the PIVA system 900 operates in conjunction with an infusion pump operating at rates of 0 to 250 mL per hour. In a related embodiment, the PIVA system 900 utilizes noise cancellation (e.g., via noise module 802) to remove the pump signal from the detected waveform (e.g., the digital signal).

In an embodiment, the PIVA system 900 displays the volume index (e.g., PIVA Score) the pulse rate, and the respiratory rate when the signal quality is adequate. For example, signal quality is adequate when the signal quality index indicates that signal quality is adequate. If signal quality is inadequate, the PIVA system 900 may indicate that the signal is of “low” quality and/or cease displaying physiological values (e.g., PR, RR, PIVA Score, and other related physiological values) so long as the signal quality remains inadequate.

The PIVA system 900 may include other additional features. In an embodiment, the PIVA system 900 includes a power supply. The power supply may be wired to an external source and/or may have internal power (e.g., a Li-ion battery). In an embodiment, the PIVA system 900 includes one or more speakers (e.g., a primary speaker and a backup speaker). The speakers may be configured to sound alarms if necessary.

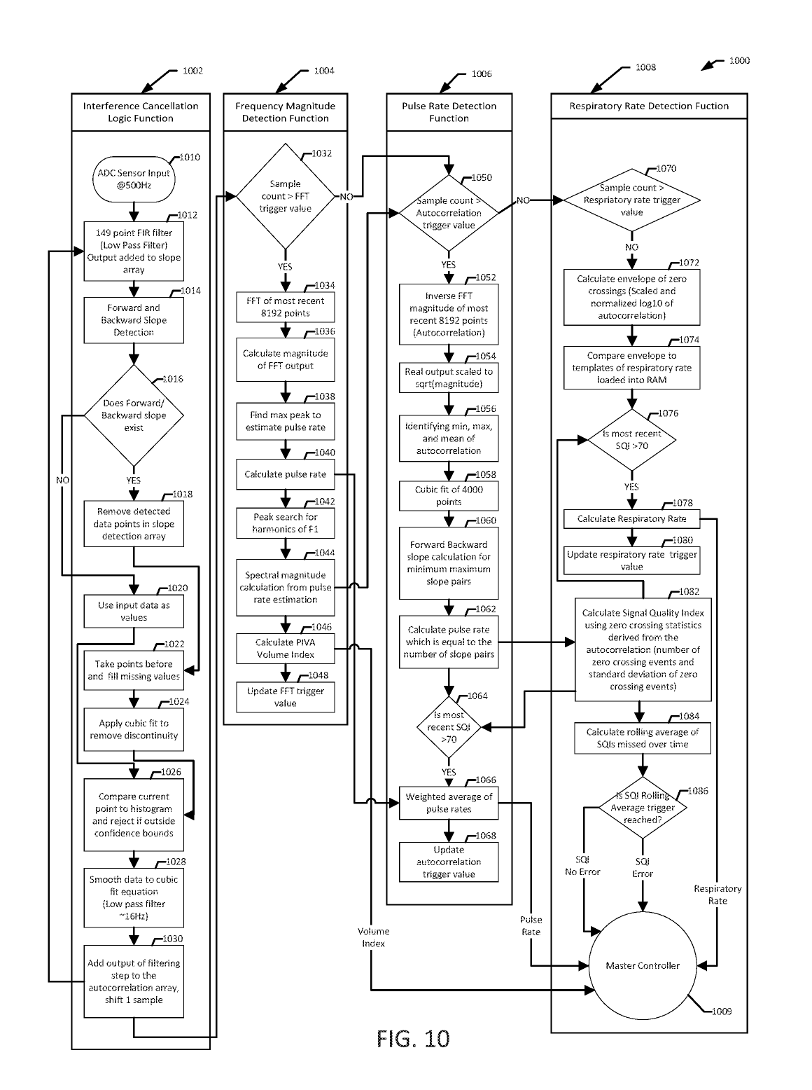

FIG. 10 illustrates another example of signal processing via process 1000. In various embodiments, any of PIVA system 100, PIVA system 900, and master controller 1009 (as detailed below) may perform process 1000. In an embodiment, process 1000 may be implemented in conjunction with process 800. In a different embodiment, example 1000 is an individual process, distinct from process 800. Although the process 1000 is described with reference to the block diagram illustrated in FIG. 10, it will be appreciated that many other configurations and methods of performing the acts associated with process 1000 may be used. For example, the order of some of the blocks may be changed, certain blocks may be combined with other blocks, and some of the blocks described may be optional.

As illustrated in FIG. 10, process 1000 may include several individual functions, including interference cancellation logic function 1002, frequency magnitude detection function 1004, pulse rate detection function 1006, and respiratory rate detection function 1008. Each of these functions may be performed by or operate with the master controller 1009 (e.g., a processor).

The interference cancellation logic function 1002 may include a sensor input (block 1010). For example, a sensor input (e.g., a pressure transducer signal) at a particular frequency (e.g., 500 Hz) may be received as the sensor input. The sensor input may be an analog and/or a digital signal. A 149 point FIR filter output (e.g., a low-pass filter) may be added to a slope array (block 1012). For example, the filter may be added to the slope array that is representative of the digital signal received as the sensor input. In an example embodiment, the interference cancellation logic function 1002 includes a low-pass convolution filter, to further improve the signal. Forward and backward slope detection may be performed (block 1014). The interference cancellation logic function may determine if the forward/backward slope exists (block 1016). If a forward/backward slope exists, detected data points in the slope detection array are removed (block 1018), missing values are filled in from the point before removal (block 1022), and a cubic-fit is applied to remove discontinuity (block 1024). Alternatively, if the forward/backward slope does not exist, input data is used as the value (block 1020).

The current point (e.g., current point of the signal) is compared to a histogram, and rejected if the current point is outside the confidence bounds (block 1026). The data is “smoothed” to a cubic fit equation (block 1028). For example, a low pass filter (e.g., 16 Hz) may be implemented. The output of the filtering step is added to an autocorrelation array, and the sample is shifted by one (block 1030). In an example, the process repeats with the 149 point FIR filter output being added to the slope array (block 1012). In a different example, the process 1000 continues on to the next function.

The frequency magnitude detection function 1004 includes determining if the sample count is greater than an FFT trigger value (block 1032).

If the sample count is greater than the FFT trigger value (block 1032), a FFT of the most recent 8192 points is performed (block 1034). The magnitude of the FFT output is calculated (block 1036). The maximum peak is identified to estimate the pulse rate (block 1038). Pulse rate is calculated (block 1040). A peak search is conducted for the harmonics of the first frequency (e.g., F₁) (block 1042). A spectral magnitude calculation is performed from the pulse rate estimation (block 1044). A PIVA Volume Index (e.g., PIVA Score) is calculated (block 1046) and the FFT trigger value is updated (block 1048). The PIVA Volume Index may be sent to the master controller 1009. Because the PIVA Volume Index is calculated, the frequency magnitude detection function 1004 may also be generally characterized as a PIVA Score function.

If the sample count is not greater than the FFT trigger value (block 1032), the process 1000 continues on to the next function. Likewise, responsive to performing the spectral magnitude calculation from the pulse rate estimation (block 1044), the process 1000 may continue on to the next function.

The pulse rate detection function 1006 includes determining if the sample count is greater than an autocorrelation trigger value (block 1050).

If the sample count is greater than the autocorrelation trigger value (block 1050), an inverse FFT magnitude of the most recent 8192 points is performed (block 1052). In an example, inverse FFT magnitude provides the time domain signal (e.g., for autocorrelation). The real output is scaled to the square-root of the magnitude (block 1054). The minimum, maximum, and mean of autocorrelation are identified (block 1056). A cubic fit of 4000 points is performed (block 1058). Forward/backward slope calculations for minimum and maximum slope pairs are performed (block 1060). Pulse rate is calculated (block 1062). In an example, calculated pulse rate is equal to the number of slope pairs. Signal quality index (SQI) is assessed, to determine if SQI is greater than a particular threshold (e.g., SQI>70). If SQI is greater than the threshold (block 1064), a weighted average of pulse rates is calculated (block 1066) and the autocorrelation trigger value is updated (block 1068). The calculated pulse rate may be sent to the master controller 1009. In an example, calculating the weighted average of pulse rates (block 1066) includes receiving input of the pulse rate calculated by the frequency magnitude detection function 1004 (block 1040).

If the sample count is not greater than the autocorrelation trigger value (block 1050), the process 1000 continues on to the next function.

The respiratory rate detection function 1008 includes determining if the sample count is greater than a respiratory rate trigger value (block 1070). If the sample count is not greater than the respiratory rate trigger value, an envelope of zero crossings is calculated (block 1072). For example, an envelope of zero crossings is scaled and normalized as log₁₀ of autocorrelation. The envelope is compared to templates of respiratory rate loaded into RAM (block 1074). Signal quality is assessed, to determine if SQI is greater than the particular threshold (e.g., SQI>70). If SQI is greater than the threshold (block 1076), respiratory rate is calculated (block 1078) and the respiratory rate trigger value is updated (block 1080). The calculated respiratory rate may be sent to the master controller 1009.

SQI is calculated using zero crossing statistics derived from the autocorrelation (e.g., the number of zero crossing events and standard deviation of zero crossing events) (block 1082). For example, calculating SQI (e.g., at block 1064 or at block 1076) may take into account pulse rate with respect to the number of slope pairs calculated by the pulse rate detection function 1006 (block 1062). The rolling average of SQIs missed over time is also calculated (block 1084). If the SQI rolling average trigger is reached (block 1086), an SQI error is sent to the master controller 1009. Likewise, if the SQI rolling average trigger is not reached (block 1086), an SQI no error is sent to the master controller 1009.

### Assessment of Patient Gait, Seizure, Activity, and Related Biometrics

As previously identified with respect to FIG. 1E, the exemplary PIVA system 100 may further include one or more additional sensors 150. These one or more additional sensors 150 may be useful, for example, to calculate other patient variables (e.g., besides PIVA Score).

More particularly, in some embodiments, the patient status metric may be directed to aspects of a patient condition, such as a patient body position or movement. Thus, information previously monitored unsystematically through observations of nurses or physicians may instead be monitored using a PVP signal on an ongoing basis. Such monitoring may include determining one or a plurality of patient status metrics associated with a position or movement of the patient, for example. In addition to patient position metrics or patient movement metrics, analysis of a PVP signal may be used to generate a plurality of patient position metrics, patient gait metrics, patient limp metrics, patient fall metrics, patient seizure metrics, other patient movement metrics, patient blood volume metrics, patient vascular response metrics, patient respiratory metrics, or other similar metrics associated with patient conditions described herein. In some embodiments, the PVP signal may be analyzed to generate primary patient metrics, such as a pulse rate, a pulse pressure, a respiratory rate, or a respiratory depth. Thus, in some embodiments, a plurality of patient status metrics may be continuously monitored solely based upon measurements related to PVP, without additional sensors or other types of measurements. By monitoring various patient status metrics using PIVA analysis of a PVP signal, the methods and systems described herein avoid the complexity, redundancy, and incompatibility of existing systems, while enabling metric-based monitoring of additional patient conditions previously monitored only through human observation.

In yet further embodiments, time-domain analysis may additionally or alternatively be performed to evaluate the PVP signal. The PVP signal generated by the pressure sensor 112 may be analyzed in the time domain or in both the time and frequency domains to determine patient status or to generate patient status metrics, such as those discussed above. This may include assessing a change in pressure signal due to fluid movement within the IV tube caused by patient movement impacting the pressure transducer in regular patterns to determine patient movement or gait. For example, the impact associated with patient gait may generate a water hammer within the IV tube 104, which may be identified by the evaluation unit 118 as a pattern of high and low pressure observations in the PVP signal. As another example, a measure of signal volatility or variance may be generated to identify seizures, where variance in the measured PVP signal will increase sharply due to pressure changes from patient movements. Thus, the analysis of the PVP signal from the sensor 112 may include identification of recurring or non-recurring patterns, which may be analyzed in either the time domain (e.g., by pattern recognition or identification of sudden changes in pressure) or in the frequency domain (e.g., by analysis of frequencies or magnitudes associated with local peaks in the frequency-domain representation of the PVP signal).

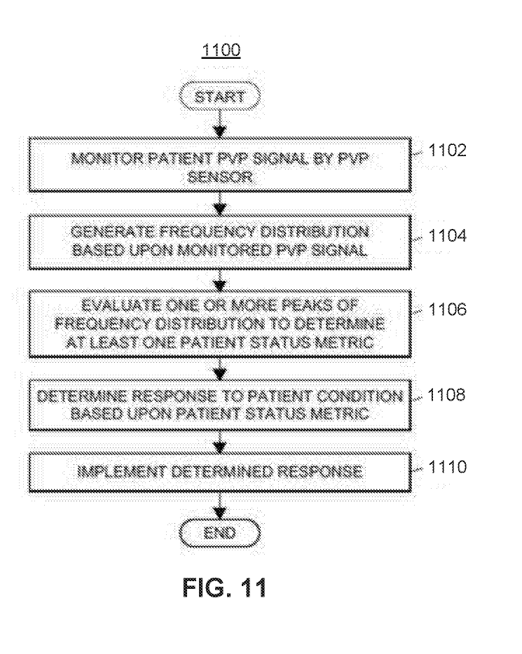

FIG. 11 illustrates a flow diagram of an exemplary patient monitoring method 1100 using patient PVP to determine and respond to patient status metrics. The exemplary patient monitoring method 1100 obtains PVP data for the patient 102 and analyzes the data using the PIVA system 100. The exemplary method 1100 may be performed by one or more software or hardware modules of the analysis component 114 using an electronic pressure signal from the pressure sensor 112, which may include generating the electronic pressure signal by the pressure sensor 112 in some embodiments. Likewise, the exemplary method 1100 may include determining and implementing responses to one or more patient metrics, which may include presenting alarms or controlling medical devices to treat a patient condition (e.g., controlling operation of a pump or other fluid source connected to the patient's circulatory system).

The exemplary method 1100 begins by monitoring a PVP signal for the patient 102 (block 1102). This may include generating a time-domain PVP signal by measuring a physical phenomenon associated with the PVP of the patient 102 using a PVP sensor (such as the pressure sensor 112 or similar sensors) via an IV tube 104. Alternatively, this may include receiving or accessing a continuous or discrete time-domain PVP signal from the PVP sensor or storage medium, which PVP signal may include raw measurement data from the sensor or data derived therefrom. However obtained, the PVP signal may be monitored until a sufficient sample period (e.g., an evaluation window) of time-domain PVP data is obtained to enable transformation and analysis, as discussed elsewhere herein.

Once the PVP signal has been obtained, the analysis component 114 may generate a frequency distribution by transforming the time-domain PVP signal to the frequency domain (block 1104). This may include applying a fast Fourier transform (FFT) to the time-domain PVP signal or other transformation techniques, as discussed elsewhere herein. As discussed elsewhere herein, the frequency distribution may be represented in any convenient form, including as an array or matrix storing data of associated frequencies and magnitudes. In some embodiments, this may include determining a plurality of frequency distributions from overlapping or non-overlapping portions of the PVP signal (e.g., a first half of the sample period and a second half of the sample period). Such plurality of frequency distributions may then be analyzed to determine one or more patient status metrics, which may then be compared to determine a change in patient status or condition.

The frequency distribution may then be analyzed by evaluating one or more frequencies (F_{N}) or associated magnitudes of peaks (P_{N}) in the frequency distribution to determine at least one patient status metric (block 1106). In some embodiments, this may include identifying the one or more frequencies (F_{N}) associated with local maxima of the frequency-domain PVP signal represented by the frequency distribution, as discussed elsewhere herein. Alternatively, the frequency distribution may include information indicating such frequencies and magnitudes. Based upon the identified frequencies (F_{N}), one or more patient metrics associated with a position or movement of the patient 102 may be determined by analyzing the frequencies or associated magnitudes. In some embodiments, a patient status metric may be determined based upon changes to a frequency or magnitude. Such changes may be determined by comparison against a previously measured frequency distribution (e.g., a frequency distribution for PVP measured during an immediately preceding sample period) or against a baseline frequency distribution (e.g., a frequency distribution for PVP measured while the condition of the patient was known, such as immediately prior to surgery). Such baseline frequency distributions may include information regarding one or more baseline frequencies and associated baseline magnitudes associated with known patient condition. The one or more patient status metrics may include patient position metrics, patient movement metrics, or primary patient metrics.

Primary patient metrics provide basic information regarding the patient 102 and may be used directly or indirectly to monitor the patient's condition. Thus, primary patient metrics may include information regarding patient circulatory and respiratory status, such as a pulse rate, a pulse pressure, a respiratory rate, or a respiratory depth. A respiratory rate or pulse rate may be determined by simply identifying the respiratory frequency (F₀) or the heart rate frequency (F₁). Harmonic frequencies (F₂, F₃, . . . F_{N}) of the heart rate frequency (F₁) may be used to identify or confirm the heart rate frequency (F₁), which may further be used to identify the respiratory frequency (F₀). Magnitudes associated with the respiratory frequency (F₀) or the heart rate frequency (F₁) may be used to determine respiratory depth or pulse pressure. In some embodiments, pulse pressure may be determined by converting the magnitude of the peak (P₁) associated with the heart rate frequency (F₁) to a time-domain signal and determining the amplitude thereof. Similarly, respiratory depth may be determined based upon the magnitude associated with the respiratory frequency (F₀) by converting the magnitude of the corresponding peak (P₀) to a signal in the frequency domain, determining the amplitude thereof, and calculating the respiratory depth based upon the amplitude. For example, the respiratory depth may be calculated using a statistical model determined from PVP measurements and respiratory depth measurements (or estimates) during a baseline period. Other similar primary patient metrics may be similarly determined from the frequency distribution.

Patient position metrics provide information regarding the posture or relative position of the parts of the patient's body. Thus, a patient position metric may indicate whether the patient 102 is in an upright position or recumbent position. Such relative position information regarding whether the patient is standing, sitting, or lying flat may be determined from the absolute or relative magnitudes of the one or more frequencies (F_{N}). For example, a patient position metric may indicate a change in an absolute magnitude of the peak (P₁) associated with the heart rate frequency (F₁) compared against a previously measured magnitude of the same peak (P₁) associated with a known patient position (e.g., sitting). Thus, an increased magnitude may indicate an increased pressure in the patient's peripheral venous system, which may indicate the patient 102 is lying flat. In some embodiments, information regarding the location of the venous access device 106 (e.g., in a patient hand, arm, or leg) may be recorded when the IV tube 104 is connected and subsequently used in the determination of the patient position metric. In further embodiments, ratios or combinations of magnitudes may be used, such as the ratio of the magnitudes of the peak (P₁) associated with the heart rate frequency (F₁) and one or more of the peaks (P₂, P₃, . . . P_{N}) associated with harmonic frequencies (F₂, F₃, . . . F_{N}) thereof.

Patient movement metrics provide information regarding the occurrence of patient movement, the type of patient movement, or the condition of the patient based upon movement. Such patient movement metrics may provide information regarding sudden patient movements, muscle spasms, patient gait, limping, stability, falls, or seizures. A patient gait metric may be determined based upon the frequency distribution associated with PVP for an ambulatory patient. A gait frequency (F_{G}) associated with the patient's gait while walking may be identified from the frequency distribution. In some embodiments, this may include first identifying the respiratory frequency (F₀) or the heart rate frequency (F₁) to identify the gait frequency (F_{G}) based upon a peak (P_{G}) in the frequency distribution that is below the heart rate frequency (F₁) and is not the respiratory frequency (F₀). In further embodiments, the heart rate frequency (F₁) may first be identified, such as by magnitude or harmonic frequencies (F₂, F₃, . . . F_{N}). Identifying the gait frequency (F_{G}) may also include a comparison of the relative magnitudes associated with the respiratory frequency (F₀) and the gait frequency (F_{G}) to identify the gait frequency (F_{G}) as being associated with a lower magnitude. In some embodiments, the respiratory frequency (F₀) may be identified based upon proximity to the respiratory frequency (F₀) of a previous time period (e.g., a prior sample period or evaluation window). The frequency and magnitude of the gait frequency (F_{G}) may be further evaluated to determine information regarding the patient's gait, such as rate, regularity, limping, or stability.

In some embodiments, a secondary gait frequency (F_{G2}) may also be identified based upon a corresponding peak (P_{G2}) in the frequency distribution. Such secondary gait frequency (F_{G2}) may be used in determining the patient gait metric or a separate patient limp metric. The secondary gait frequency (F_{G2}) may be identified as a fraction or multiple of the gait frequency (F_{G}). Alternatively, the secondary gait frequency (F_{G2}) may be identified as beginning and ending concurrently with the gait frequency (F_{G}) across multiple frequency distributions associated with a time series of sample periods. As another alternative, the secondary gait frequency (F_{G2}) may be identified as beginning associated with a corresponding peak (P_{G2}) in the frequency distribution that is not associated with the respiratory frequency (F₀), the heart rate frequency (F₁), a harmonic frequency (F₂, F₃, . . . F_{N}), or the gait frequency (F_{G}). In some related embodiments, a minimum threshold magnitude may be used to ensure the secondary gait frequency (F_{G2}) has a sufficient magnitude relative to the gait frequency (F_{G}), thereby eliminating from consideration minor frequency peaks caused by noise or other phenomena not related to patient perambulation. However identified, the secondary gait frequency (F_{G2}) may be evaluated to determine the consistency of the patient's gait, including whether the patient is limping while walking. The regularity (i.e., fixedness of the frequency) or the magnitude associated with the secondary gait frequency (F_{G2}) may be evaluated to determine a patient gait consistency metric (which may be part of the patient gait metric) indicating whether the patient's gait is stable, unstable, normal, or abnormal (i.e., indicating a limp). For example, the consistent location of the secondary gait frequency (F_{G2}) at an integer fraction or multiple of the gait frequency (F_{G}) may indicate a limp, whereas shifting frequency values of the secondary gait frequency (F_{G2}) over time may indicate instability. Similarly, a larger magnitude associated with the secondary gait frequency (F_{G2}) relative to the magnitude associated with the gait frequency (F_{G}) may indicate a more pronounced limp.

Although only one secondary gait frequency (F_{G2}) is discussed above, it should be understood that multiple secondary gait frequencies (F_{G2}) could be identified and evaluated to determine the consistency of the patient's gait. Similarly, in some embodiments, the magnitude associated with the gait frequency (F_{G}) may be compared against a plurality of magnitudes associated with other frequencies within the frequency distribution to determine the consistency of the patient's gait. Such comparison may be made against a measure of the total or average magnitude across a range of frequencies of the frequency distribution. For example, the ratio of the magnitude associated with the gait frequency (F_{G}) to the median magnitude of the frequency distribution may be calculated as a patient gait metric indicating stability of an ambulatory patient. A higher ratio indicates a steady gait while the patient is walking, while a lower ratio indicates instability as minor variations in gait lead to relatively greater magnitudes at other frequencies. Thus, even frequencies not associated with peaks (i.e., local maxima) of the frequency distribution may be evaluated in generating some patient status metrics, particularly metrics associated with stability or instability. In some embodiments, a separate patient stability metric may be determined based upon magnitudes associated with a plurality of secondary gait frequencies (F_{G2}) or other frequencies, including averages (e.g., medians) across a range of frequencies within the frequency distribution.

Patient movement metrics may further include patient fall metrics indicating a patient has fallen. In some embodiments, the patient fall metric may be determined as a binary metric indicating either presence or absence of a fall. Alternatively, the patient fall metric may be determined as a probability of a fall based upon the frequency distribution. In the time domain, a fall will appear as a sudden spike in measured pressure, with PVP rising quickly as the shock of impact propagates through the circulatory system and then quickly returning approximately to previous levels. In the frequency domain, such a spike or pulse in the time domain signal is identifiable by a characteristic pattern of peaks and troughs. For example, the spike may be viewed as approximating a square pulse, the characteristic frequency distribution of diminishing magnitude peaks symmetrically around frequency zero (0 Hz) is well known. Thus, the PVP pulse associated with the impact when a patient falls may be identified by identifying a pattern within the frequency distribution that is associated with a short-duration pulse in the time-domain PVP signal. In some embodiments, the pattern may be identified after identifying and removing peaks in the frequency distribution associated with frequencies of interest, such as the respiratory frequency (F₀), the heart rate frequency (F₁), the harmonic frequencies (F₂, F₃, . . . F_{N}) thereof, or the gait frequency (F_{G}). In alternative embodiments, the time-domain spike may be identified as a large magnitude of a peak associated with a low frequency in the frequency distribution. As the transient pressure pulse from falling will be large relative to other influences on the time-domain PVP signal, the magnitude associated with the primary peak of the frequency distribution generated thereby will also be large. Thus, a fall may be detected in some instances based upon such magnitude.

In further embodiments, the spike associated with a fall may further be identified in the time-domain PVP signal, which may be advantageous in confirming the occurrence of the fall and identifying the time of the fall. Once the time of the fall is identified, the sample period including the fall may be divided into a pre-fall portion and a post-fall portion for further evaluation. In some instances, the pre-fall and post-fall portions of the sample period may be augmented by the addition of earlier and later values of the time-domain PVP signal, respectively, to ensure sufficient time-domain PVP data for evaluation of each portion of the original sample period. The pre-fall and post-fall portions may be separately transformed to generate pre-fall and post-fall frequency distributions. Shifts in frequency or changes in magnitude of the peaks of interest may then be evaluated to determine a severity of the fall, which may be included in a patient fall metric. For example, a percentage increase in the heart rate frequency (F₁) following the fall may be calculated as a metric to evaluate severity of the fall, as the patient's body responds to the incident. Other similar changes in frequencies or associated magnitudes may likewise be determined in various embodiments.

Patient movement metrics may further include patient seizure metrics indicating occurrence of a seizure. The patient seizure metrics may include a ratio of the magnitude associated with the heart rate frequency (F₁) relative to magnitudes associated with one or more other frequencies within the frequency distribution. For example, the ratio of the magnitude associated with the heart rate frequency (F₁) relative to the average magnitude of the frequencies within a range of the frequency distribution (e.g., from 0 Hz to 5 Hz) may be used as a patient seizure metric to indicate how well-defined the heart rate is relative to other components of the time-domain PVP signal. Although other factors may affect it, the ratio between the magnitude of the heart rate frequency (F₁) and the average magnitude will be less for a seizing patient than for a healthy patient. During a seizure, movements of the patient's body generate substantial noise in the PVP signal, resulting in a general increase in magnitudes associated with frequencies across the frequency distribution. If sufficiently severe, the heart rate frequency (F₁) may not be identifiable from the surrounding noise. In further embodiments, the patient seizure metric may be determined based upon an absolute level of the average magnitude of the frequency distribution or the average (e.g., median) magnitude from a sample of a plurality of frequencies (e.g., ten or twenty frequencies). In related embodiments, the patient seizure metric may be determined based upon a comparison of an average magnitude between frequency distributions associated with different sample periods, such that a sharp increase in average magnitude may be indicative of a seizure.

In some embodiments, the analysis component 114 may simultaneously monitor a plurality of patient status metrics, such as by evaluating the frequencies (F_{N}) or associated magnitudes of peaks (P_{N}) in the frequency distribution. Such patient status metrics may be determined using the same frequency distribution for the same sample period. When a comparison between sample periods is used to generate a patient status metric, the same plurality of frequency distributions associated with the same sample periods may be used. The plurality of patient status metrics may include metrics from within one or more of the primary patient metrics, patient position metrics, or patient movement metrics groups discussed above, as well as other metrics. For example, a patient seizure metric and another patient movement metric (e.g., a patient gait metric or a patient fall metric) may be monitored simultaneously from the same frequency distribution. As another example, a primary patient metric (e.g., a pulse rate, a pulse pressure, a respiratory rate, or a respiratory depth) and a patient position metric or a patient movement metric may be monitored simultaneously from the same frequency distribution. As yet another example, a fall or seizure may be identified by identifying combinations of abnormal gait metrics (e.g., variable gait frequencies, secondary gait frequencies, or a water hammer effect) with patient stress indicators (e.g., increased heart rate or respiratory rate). By using (explicitly or implicitly) the frequency and magnitude information associated with the observed PVP signal, any or all of the foregoing patient status metrics may be monitored without requiring the use of additional sensors beyond the PVP sensor (e.g., the pressure sensor 112).

Although the foregoing description has presented the analysis as being performed using frequency-domain PVP data, other embodiments may additionally or alternatively include other types of analysis to generate patient status metrics, including any of the primary patient metrics, patient position metrics, and patient movement metrics discussed above or combinations thereof. For example, a patient movement metric may be determined by analyzing the PVP signal in the time domain to identify physical movement or gait of the patient by assessing the change in pressure due to fluid movement within the IV tube (e.g., water hammer effect) caused by patient arm movement within the gait activity, impacting the pressure sensor 112 in regular patterns. As another example, a patient fall metric may be determined by identifying pressure spikes beyond a threshold magnitude in the time-domain PVP signal from the pressure sensor 112.

Based upon the one or more patient status metrics, the analysis component 114 may determine a response to a patient condition (block 1108) and implement the determined response (block 1110). For example, the response unit 116 may determine whether a response is required and cause any required response to be implemented. This may include determining one or more patient conditions by evaluating one or more patient status metrics. Patient conditions may include a position (e.g., sitting or standing), instability, limp, fall, seizure, or other similar conditions. The patient conditions may include position conditions, movement conditions, or primary conditions. For example, position conditions may include lying, sitting, or standing, while movement conditions may include walking, unsteady walking, limping, falling, or seizing. Primary conditions may include shallow breathing, hyperventilating, not breathing, irregular breathing, normal breathing, normal heartbeat, slow heartbeat, rapid heartbeat, or irregular heartbeat. Determining each of the patient conditions may include evaluating one or more patient status metrics. For example, determining a patient is unsteadily walking may include evaluating a patient gait metric to determine whether the patient is walking, then evaluating a separate stability metric to determine the patient is unstable while walking. Some conditions may be determined based upon a combination of such metrics. For example, determining a patient is in a normal condition may require all monitored patient status metrics to be within acceptable ranges.

Whether the patient condition is determined based upon the patient status metrics or the patient condition is implied from the value of a patient status metric, one or more responses related to one or more patient conditions may be determined based upon the patient status metrics. Although some conditions may require active responses, other conditions may simply require continued monitoring (or no response). For example, when all patient status metrics that have been determined for the patient 102, the analysis component 114 may determine that the appropriate response to the normal patient condition is to continue monitoring. In such case, the response may be implemented by generating or obtaining additional sensor data regarding PVP and performing further analysis on the additional data according to the methods described herein. Active responses may include presenting an alarm or controlling operation of a medical device. An alert may be generated based upon the patient status metrics or conditions determined therefrom, which alert may include information regarding the condition or remedial actions to be taken. For example, an alert may indicate that a patient is unsteadily walking. A visual, audible, or tactile alarm or warning may be presented to appropriate personnel (e.g., via the monitor 120) based upon the alert, which may include displaying a message indicating the type of condition or a recommended course of action. Operation of a medical device in response to the patient condition may include controlling the fluid source 110 to adjust fluid flow to the patient 102. This may include adjusting a flow rate, starting or stopping fluid flow, adding one or more drugs to the fluid, or similar control actions, as discussed further below. In some embodiments, the analysis component 114 may directly control the implementation of the response by controlling the fluid source 110 or the monitor 120. Alternatively, the analysis component 114 may communicate control information to other devices to cause those devices to present an alert or control operation of a medical device.

In some embodiments, the patient condition or the response to the patient condition may be determined based in part upon additional sensor data from one or more additional sensors 150. For example, a pressure sensor may generate additional sensor data indicating whether the patient is in a bed, which may be combined with patient movement metrics to determine whether the patient is at risk to fall. If the additional sensor data indicates patient is lying in the bed, no response beyond continuing monitoring may be required despite a patient stability metric indicating instability. If the additional sensor data instead indicates the patient is not lying in the bed, however, an alarm may be generated to alert appropriate personnel that the patient is at risk of falling. Some embodiments may not include additional sensors 150 or may not use sensor data therefrom to determine patient conditions or responses to the patient conditions. In such embodiments, responses may be determined using only the patient status metrics derived from measurements of PVP via the pressure sensor 112.

As used in this specification, including the claims, the term “and/or” is a conjunction that is either inclusive or exclusive. Accordingly, the term “and/or” either signifies the presence of two or more things in a group or signifies that one selection may be made from a group of alternatives.

The many features and advantages of the present disclosure are apparent from the written description, and thus, the appended claims are intended to cover all such features and advantages of the disclosure. Further, since numerous modifications and changes will readily occur to those skilled in the art, the present disclosure is not limited to the exact construction and operation as illustrated and described. Therefore, the described embodiments should be taken as illustrative and not restrictive, and the disclosure should not be limited to the details given herein but should be defined by the following claims and their full scope of equivalents, whether foreseeable or unforeseeable now or in the future.

The invention is claimed as follows:

1. A system for monitoring a patient using a measurement associated with a peripheral venous pressure (PVP) within a peripheral vein of a circulatory system of the patient while the circulatory system of the patient is connected to a pump, comprising:
a PVP sensor including a transducer disposed adjacent to or connected to an intravenous (IV) tube in fluid connection with the peripheral vein and configured to generate an electronic signal associated with the PVP while the circulatory system of the patient is connected to the pump; and
an evaluation unit, including a computer processor communicatively connected to the PVP sensor to receive the electronic signal and a memory storing non-transitory computer-readable instructions that, when executed by the computer processor, cause the evaluation unit to:
obtain a time-domain PVP signal comprising values of an electronic signal associated with the PVP from the transducer based upon a physical phenomenon associated with the PVP of the patient over a sample period, wherein the sample period includes a plurality of time segments, including (i) one or more active time segments during which the pump is operating and (ii) one or more inactive time segments during which the pump is not operating;
identify a first plurality of the values of the time-domain PVP signal associated with the one or more inactive time segments and a second plurality of the values of the time-domain PVP signal associated with the one or more active time segments, based upon evaluation of the values of the time-domain PVP signal;
generate a filtered time-domain PVP signal based upon the first plurality of the values and excluding the second plurality of the values;
apply a transformation to the filtered time-domain PVP signal to generate a frequency-domain PVP signal; and
determine a patient status metric for the patient based upon the frequency-domain PVP signal.

2. The system of claim 1, wherein the pump is a peristaltic IV pump.

3. The system of claim 1, wherein the pump is configured to operate periodically, such that the one or more active time segments and the one or more inactive time segments periodically alternate.

4. The system of claim 1, wherein the IV tube is disposed between the patient and the pump such that a part of the pump is in fluid connection with the peripheral vein of the circulatory system of the patient via the IV tube.

5. The system of claim 4, wherein:
the transducer comprises a pressure sensor disposed in fluid connection with an interior of the IV tube; and
the physical phenomenon associated with the PVP is a pressure within the interior of the IV tube.

6. The system of claim 4, wherein the instructions further cause the evaluation unit to:
determine whether the patient status metric indicates a condition of the patient is abnormal; and
adjust operation of the pump when the patient status metric indicates the condition of the patient is abnormal by changing a rate of flow of a fluid from the pump into the circulatory system of the patient.

7. The system of claim 1, wherein the executable instructions that cause the evaluation unit to generate the filtered time-domain PVP signal include instructions that cause the evaluation unit to remove the one or more active time segments from the time-domain PVP signal.

8. The system of claim 7, wherein the executable instructions further cause the evaluation unit to generate the filtered time-domain PVP signal by, for each of one or more pairs of the active time segments:
identifying one or more corresponding values within both of the active time segments of the pair; and
combining the active time segments of the pair by aligning the one or more corresponding values within both of the active time segments of the pair.

9. The system of claim 1, wherein the executable instructions that cause the evaluation unit to generate the filtered time-domain PVP signal include instructions that cause the evaluation unit to:
estimate a third plurality of values as substitute values for the one or more active time segments, wherein the third plurality of values are estimated based upon the first plurality of values without reference to the second plurality of values; and
generate the filtered time-domain PVP signal by combining the first plurality of values for the inactive time segments and the third plurality of values for the active time segments.

10. The system of claim 9, wherein the third plurality of values are estimated by performing at least one of regression analysis, forward-backward slope calculation, two-sided slope detection, and mirror matched filtering on at least the first plurality of values.

11. The system of claim 1, wherein the executable instructions that cause the evaluation unit to determine the patient status metric include instructions that cause the evaluation unit to:
identify a plurality of frequencies associated with local maxima of the frequency-domain PVP signal; and
determine the patient status metric based at least in part upon at least one of the plurality of frequencies associated with the local maxima.

12. The system of claim 1, wherein the patient status metric is a blood volume metric indicating one or more of the following: hypovolemia, hypervolemia, or euvolemia.

13. A device for monitoring a patient, comprising:
a peripheral venous pressure (PVP) sensor, including a transducer configured to monitor a physical phenomenon associated with a PVP within a peripheral vein of a circulatory system of the patient while the circulatory system of the patient is connected to a pump; and
an evaluation unit, including a computer processor communicatively connected to the PVP sensor and a memory storing non-transitory executable instructions that, when executed by the computer processor, cause the evaluation unit to:
obtain a time-domain PVP signal comprising values of an electronic signal associated with the PVP received from the transducer of the PVP sensor over a sample period, wherein the sample period includes a plurality of time segments, including (i) one or more active time segments during which the pump is operating and (ii) one or more inactive time segments during which the pump is not operating;
identify a first plurality of the values of the time-domain PVP signal associated with the one or more inactive time segments and a second plurality of the values of the time-domain PVP signal associated with the one or more active time segments, based upon evaluation of the values of the time-domain PVP signal;
generate a filtered time-domain PVP signal based upon the first plurality of the values and excluding the second plurality of the values;
apply a transformation to the filtered time-domain PVP signal to generate a frequency-domain PVP signal; and
determine a patient status metric for the patient based upon the frequency-domain PVP signal.

14. The device of claim 13, wherein:
the time-domain PVP signal comprises a first time series of discrete values;
the filtered time-domain PVP signal comprises a second time series of discrete values; and
the second time series contains at least one segment of a sequential plurality of values within the second time series that are equivalent to a corresponding segment of a sequential plurality of corresponding values within the first time series.

15. The device of claim 13, wherein the executable instructions that cause the evaluation unit to generate the filtered time-domain PVP signal include instructions that cause the evaluation unit to remove the one or more active time segments from the time-domain PVP signal.

16. The device of claim 13, wherein the executable instructions that cause the evaluation unit to generate the filtered time-domain PVP signal include instructions that cause the evaluation unit to:
estimate a third plurality of values as substitute values for the one or more active time segments, wherein the third plurality of values are estimated based upon the first plurality of values without reference to the second plurality of values; and
generate the filtered time-domain PVP signal by combining the first plurality of values for the inactive time segments and the third plurality of values for the active time segments.

17. A method of monitoring a patient using a measurement associated with a peripheral venous pressure (PVP) within a peripheral vein of a circulatory system of the patient while the circulatory system of the patient is connected to a pump, comprising:
monitoring, by a transducer, a physical phenomenon associated with the PVP of the patient over a sample period, wherein the sample period includes a plurality of time segments, including (i) one or more active time segments during which the pump is operating and (ii) one or more inactive time segments during which the pump is not operating;
obtaining, by a processor of an evaluation unit, a time-domain PVP signal comprising values of an electronic signal associated with the PVP from the transducer based upon the monitored physical phenomenon over the sample period;
identifying, by the processor of the evaluation unit, a first plurality of the values of the time-domain PVP signal associated with the one or more inactive time segments and a second plurality of the values of the time-domain PVP signal associated with the one or more active time segments, based upon evaluation of the values of the time-domain PVP signal;
generating, by the processor of the evaluation unit, a filtered time-domain PVP signal based upon the first plurality of the values and excluding the second plurality of the values;
applying, by the processor of the evaluation unit, a transformation to the filtered time-domain PVP signal to generate a frequency-domain PVP signal; and
determining, by the processor of the evaluation unit, a patient status metric for the patient based upon the frequency-domain PVP signal.

18. The method of claim 17, wherein generating the filtered time-domain PVP signal includes removing the one or more active time segments from the time-domain PVP signal.

19. The method of claim 17, wherein generating the filtered time-domain PVP signal includes:
estimating a third plurality of values as substitute values for the one or more active time segments, wherein the third plurality of values are estimated based upon the first plurality of values without reference to the second plurality of values; and
generating the filtered time-domain PVP signal by combining the first plurality of values for the inactive time segments and the third plurality of values for the active time segments.

20. The method of claim 17, wherein the third plurality of values are estimated by performing at least one of regression analysis, forward-backward slope calculation, two-sided slope detection, and mirror matched filtering on at least the first plurality of values.
